(19) United States
(12) Patent Application Publication
(10) Pub. No.: US 2008/0026486 A1
(43) Pub. Date: Jan. 31, 2008

(54) Assay Methods, Materials and Preparations

(76) Inventors: Matthew Cooper, Cambridge (GB); Xin Li, Shanghai (CN)

Correspondence Address: MORGAN LEWIS & BOCKIUS LLP, 1111 PENNSYLVANIA AVENUE NW, WASHINGTON, DC, 20004, US

(21) Appl. No.: 11/662,081

(22) PCT Filed: Sep. 8, 2005
(86) PCT No.: PCT/GB05/03455

§ 371 Date: Aug. 2, 2007

(30) Foreign Application Priority Data
Sep. 9, 2004 (GB) 0420062.2

(51) Int. Cl. C08B 37/00; A61K 41/00; G01N 33/543

(52) U.S. Cl. 436/518; 428/426; 428/457; 428/532; 528/373

(57) ABSTRACT

Disclosed is a polymer comprising covalently bound side chains of the formula —X—Y—Z—R wherein X is a spacer group; is a sulphur, selenium or tellurium atom; Z is a sulphur, selenium or tellurium atom any of which may be bonded to one or two oxygen atoms; and wherein R is any suitable moiety such that —Z—R constitutes a leaving group.

### FIELD OF THE INVENTION

The present invention relates to novel polymers, their preparation and their use in coating surfaces, to the coated surfaces and to their use in assay devices and methods. More particularly this invention relates to polymers containing moieties incorporating chalcogen groups, to their preparation and their use in coating surfaces in biosensors, to the biosensors themselves and to their use in assay methods.

### BACKGROUND TO THE INVENTION

The immobilisation of molecules at surfaces in a specific manner while minimising non-specific binding has been shown to be important in many fields in which biocompatibility is a factor, for example in the preparation of biosensors; and the prevention of surface fouling. Such immobilisation has been achieved with varying degrees of success by using biocompatible coatings, which can be coupled to a member of a specific binding pair. Under the appropriate conditions, the other member of the pair can be then become bound to the coating under assay conditions while non-specific binding, (for example unwanted binding of proteins, cells, bacteria or other unwanted materials) is minimised. This has enabled the use of the coated materials in biosensors, in particular surface plasmon resonance and piezo-electric sensing devices.

Examples of the methods used to date to provide coatings include passive adsorption of proteins on polystyrene as in conventional ELISA; silanisation of glass with polyethylene substituted by silyl groups and by terminal capture groups (Ref. 1); passive adsorption of polylysine on glass and subsequent adsorption of DNA and polyethylene glycol with terminal functional groups (Refs. 2 and 3); passive adsorption of vesicles and Langmuir-Blodgett layers onto glass, self-assembled monolayers (SAMs) or silane layers (Ref. 4); passive adsorption of proteins or peptides on cellulose nitrate or the like as in conventional dot blots; solvent casting of cellulose nitrate or the like onto metal (Ref. 5); electrostatically adsorbed coatings such as graft copolymers based on polylysine used to immobilise proteins on oxides such as those of tantalum, niobium, titanium and silicon (Refs. 6, 7,and 8).

Other methods include self-assembled monolayers (SAMs). Substrates have been decorated with oligo- and poly-ethylene glycol n-alkanethiolates that form SAM coatings on metals. These oligo-ethylene glycol containing SAMs are prone to oxidation and, being planar, provide only a limited scaffold to which materials can be attached so limiting the signal strength that can be achieved (Refs. 9 and 10). This approach has been used, despite these disadvantages, to determine the binding of low molecular weight drug candidates to certain receptors (Ref. 11).

The use of a SAM to which a biocompatible porous matrix can be attached for application to a surface plasmon resonance biosensor is also known (Ref. 12). In this case an n-alkanethiol SAM with an omega functionalised hydroxyl group was activated by an epoxy group for attachment of carboxymethylated dextran.

It is also known to use SAMs to which a porous matrix is attached for use in acoustic sensors (Refs. 13 and 14).

It is known to use the thiol groups of mercaptoethyl carbamoyl dextran to anchor a polymer to gold or silver. It was found that surface density was dependent inversely on polymer molecular weight and dependent directly on side chain density. No discussion of the amount of functionality or residual thiol groups was given in terms of chemically attaching receptors (Ref. 16). Siloxane backbones with disulphide side chains to anchor to gold are known together with polymers with polyethylene glycol side chains terminating in reactive esters for immobilising receptors (Ref. 16). Polymethyl-methacrylate having sulphide containing side chains to anchor to a surface and having further reactive groups for the attachment of receptors are also known. Residual thiol groups were said to be capable of further reaction, but no specific guidance was given as to how this could be achieved (Ref. 17).

Thiolated polyvinylalcohol containing residual SH groups have been contemplated for use in a biosensor although without stating how attachment is to be achieved (Ref. 18). Thiol derivatised polystyrene has been investigated and was found to be less effective because the leaving group had to be accounted for (Ref. 19). Acrylic polymers derivatised with C11—SS—C₅H₁₁ side chain anchor groups were said to self-assemble. Although stability was improved over monomer films they were found to be less organised than monomeric analogues (Ref. 20). Other SAM acrylic polymers containing dithioalkyl side chains are also known (Ref. 21).

Growing polymers onto SAM coated areas has been described (Ref. 22) as have brush polymers terminally anchored to sensing surfaces using silanes (Ref. 24). Topical compositions, for example for treating amino based substrates for cosmetic uses, comprising protected thiol compounds have been described (Ref. 24). Biosensors have been employed which have coupled dextrans to surfaces using thiol groups (Ref. 25).

Sulfones, sulfoxides, selenones, selenoxides and higher oxidation state chalcogenides per se are known (Refs. 26-31) but they have not been used in connection with assay devices such as biosensors or in coating surfaces with polymers, for example polymers to which a member of a binding pair is linked.

Such frequent attempts in the art to provide enhanced materials demonstrate the continuing need for further improvements in polymers, which can be used to coat surfaces and, in particular, metal surfaces that are prone to non-specific binding or fouling. Such new polymers should also preferably be able to bond a member of a specific binding pair so that they can be used in biosensors to determine the presence and/or properties of the other member of the specific binding pair. In addition, they should avoid the difficulties associated with known leaving groups used to attach polymers to metals such as the SH and S-lower alkyl groups and the like.

Prior art attempts to employ sulfydryl groups have suffered from a tendency to oxidation with ambient or dissolved oxygen. This can occur during purification, storage or use and can change the reactivity of the polymer to the metal surface as —SH groups convert to —SS— groups. This can also cause cross-linking of the polymer with the result that a more viscous, gel-like structure is formed which is less suitable for use in biosensors. Prior art use of disulfide containing polymers has resulted in the generation of short alkyl chain moieties arising from the severing of the S—S bond. These can become adsorbed onto the metal surface reducing the number of active sites available for polymer binding. This can also reduce the degree of control over the polymer adsorption which affects the function of the polymer coating, and can render the metal coating hydrophobic through attachment of the alkyl chain moieties, which promotes non-specific binding and fouling.

The present invention aims to reduce or overcome one or more of these difficulties in the prior art and to provide polymers that allow for a good and firmly fixed loading on a metal substrate. In addition they can show lower non-specific binding and allow specific binding of a member of a specific binding pair so that they may be used in biosensors for the determination of the presence and/or properties of the other member of the specific binding pair.

In addition, the invention allows for the formation of a non-planar three-dimensional matrix to which a member of a specific binding pair can be attached, thereby increasing the amount of the member which can be presented for a given area of surface, which in turn increases the amount of the other member of the pair that can be captured. This invention also has the effect of reducing the degree of non-specific binding to the surface, by effectively masking the chemical and physical properties of the surface. This is particularly important in the case of metal surfaces.

### BRIEF DESCRIPTION OF THE INVENTION

This invention provides a polymer which has covalently bound side chains of the formula —X—Y—Z—R wherein X is a spacer group; Y is a sulphur, selenium or tellurium atom; Z is a sulphur, selenium or tellurium atom, any of which may be bonded to one or two oxygen atoms; and wherein R is any suitable moiety such that —Z—R constitutes a leaving group.

The polymer may be reacted with a surface, preferably a metal surface, so that it becomes bound to the surface by displacement of some of the —Z—R groups. This is then reacted with a compound H—Z—R1 (where R1 is a member of a specific binding pair) which displaces some or all of the residual —Z—R groups, thereby indirectly anchoring the R1 moiety to the surface. Another option is to add the R1 moiety to another reactive group present elsewhere (e.g. not necessarily in the side chains) in the polymer.

In an alternative embodiment, in addition to the side chains of the formula —X—Y—Z—R, the polymer may also comprise side chains of the formula —X—Y—Z—R1 (where R1 is a member of a specific binding pair), the other member of the specific binding pair being an analyte. The polymer may be reacted with a surface, preferably a metal surface, so that it becomes bound to the surface by displacement of —Z—R groups; thus, in essence there are two ways in which the polymer of the invention may be utilised in a biosensor:
(i) The polymer may be reacted with a surface, so that it becomes bound thereto by displacement of at least some of the —Z—R groups. A member of a specific binding pair may then be joined to the bound polymer, typically by reaction with remaining unreacted —Z—R groups.
(ii) Alternatively, the polymer may be formed so as to comprise a member of a specific binding pair prior to its immobilisation on a surface for example, the polymer may comprise a mixture of —X—Y—Z—R and —X—Y—Z—R1 groups, where R1 is a member of a specific binding pair. The polymer, containing the member of the specific binding pair, is then immobilised on a surface by displacement of —Z—R groups.

Using either approach (i) or (ii) the surface becomes coated with a polymer that has —X—Y—Z—R1 side chains. The coated surface may be used in biosensors such as those employing surface plasmon resonance or piezo-electric sensing in order to analyse a sample e.g. for the presence of the other member of the specific binding pair.

The polymers of the present invention can be used for many different purposes, to coat biosensors or other objects. In particular, in non-biosensor contexts, the polymers (especially hydrophilic and/or neutral polymers) in accordance with the invention can be used to enhance biocompatibility and/or prevent non-specific binding or fouling. Such characteristics could be especially useful in medical or surgical implants and prosthetic devices, or in the formation of anti-fouling coatings on delicate or expensive pieces of equipment in environments where fouling (e.g. due to non-specific binding) could be problematic. The surfaces to be coated with polymers of the invention may be planar or non-planar. In particular, in addition to use in biosensors, the polymers may be used to coat particulate solids, such as micro- or nanoparticulates, especially metallic nanoparticles. Another use of the polymers of the invention is in lithographic applications: polymers in accordance with the invention can be deposited onto a surface to form an electrically insulating pattern or layer.

### DETAILED DESCRIPTION OF THE INVENTION

The present invention provides a polymer which has covalently bound side chains of the formula —X—Y—Z—R wherein X is a spacer group; Y is a sulphur, selenium or tellurium atom; Z is a sulphur, selenium or tellurium atom any of which may be bonded to one or two oxygen atoms; and wherein R is any suitable moiety such that —Z—R constitutes a leaving group.

Favourably R is a moiety such that the conjugate acid HZR has a pKa of less than 8 and preferably less than 6, more preferably less than 4.

In such polymers favoured values for Y are S and Se of which S is particularly apt. In such polymers favoured values for Z include S, SO and SO₂ of which S and SO₂ are particularly apt.

In such polymers preferred values for —Y—Z— include S—S and S—SO₂ of which S—S is particularly preferred.

Apt values for R when Z is a S, Se or Te atom, and preferably a S atom, include unsaturated groups conjugated to electron withdrawing groups, aromatic groups and heteroaromatic groups and electrophilic groups. Suitable electron withdrawing groups include lower alkyloxycarbonyl, nitrile, nitro, lower alkylsulphonyl, trifluoromethyl and the like. Suitable aromatic groups include optionally substituted phenyl where there are up to three substituents selected from nitro, trifluoromethyl, nitrile, lower alkyloxycarbonyl, or other electron withdrawing groups. (Throughout the present specification the term “lower” is used to mean containing up to 6 carbon atoms, more aptly 1-3 carbon atoms and favourably 1 or 2 carbon atoms, unless the context clearly dictates otherwise). Suitable heteraromatic groups include those of 5- or 6-membered rings optionally fused to the residue of phenyl ring or a further 5- or 6 membered heteroaromatic ring. Said heteroaromatic group may be optionally substituted by one or two lower alkyl, phenyl, ═O, ═S, trifluoromethyl, nitro or nitrile groups.

Particularly apt groups —S—R include those derived from aromatic thiols and heteraromatic thiols or their thione tautomer. Particularly suitable —S—R groups include those derived from imidazole; pyrrolidine-2-thione; 1,3-imidasolidine-2-thione; 1,2,4-triazoline-3(5)-thione; 1,2,3,4-tetrazoline-5-thione; 2,3-diphenyl-2,3-dehydrotetrazolium-5-thione; N(1)-methyl-4-mercaptopiperidine; thiomorphyline-2-thione; thiocaprolactam; pyridine-2-thione; pyrimidine-2-thione; 2-thiouracil; 2,4-dithiouracil; 2-thiocytosine; quinoxazoline-2,3-dithione; 1,3-thiazoline-2-thione; 1,3-thiazolidine-2-thione; 1,3-thiazolidine-2-thione-5-one; 1,3,4-thiadiazoline-2,5-dithione; 1,2-oxazolidine-2-thione; benz-1,3-oxazoline-2-thione; 1,3,4-oxadiazoline-2-thione and analogues in which the sulphur is replaced by selenium or tellurium.

A particularly favoured R group is the 2-pyridyl group (2-Py).

A preferred —Z—R group is the —S-2-pyridyl group.

A preferred —Y—Z—R group is the —S—S-2-pyridyl group.

The spacer group —X— will aptly be of the formula —A—B—. The nature of group —A— will depend upon the group in the polymer to which the side chain will be attached. The most common groups of the polymer to which the side chain will be attached are CO₂H, OH and optionally mono lower alkyl substituted NH₂.

Other suitable groups present in the polymer which may be utilised for attaching the side chain include those used for immobilisation in liquid chromatography such as aldehyde, hydrazide, carboxyl, epoxy, vinyl, phenylboronic acid, nitrile-triacetic acid, imidodiacetic acid and the like.

In the case of polymers containing carboxyl groups the side chain may be attached via an ester group —CO—O—B—. Thus the group —A— represents an oxygen atom attached to the residual carboxyl group of the original carboxyl group. In an alternative the side chain may be attached via an amide group where the group —A— is an —NH— group or lower alkyl substituted —NH— group.

In the case of polymers containing hydroxy groups, the side chain may be attached via an acylated hydroxy group —O—CO—B—. Thus the group —O—X— may represent a —O—CO—B—, —O—CO—O—B—, —O—CO—NH—B— or lower alkylated —O—CO—NH—B— group.

In the case of polymers containing primary or secondary amino groups, the side chain may be attached in an analogous manner to the case for hydroxy containing polymers. Thus the group —NH—X— or its lower alkyl substituted derivative may represent —NH—CO—B—, —NH—CO—O—B—, —NH—CO—NH—B— or their lower alkyl substituted derivatives.

The other suitable groups referred to above may be used for attachment of side chains in manners appropriate to their chemistry as will be understood by the skilled chemist.

The group B may be any convenient group such as an alkylene, phenyl or like group which may be unsubstituted or substituted by lower alkyloxy, halo, oxo, trifluoromethyl, nitrile or other group that does not interfere with the formation and use of the —Y—Z—R moiety.

Particularly apt groups B include lower alkylene groups optionally interrupted by an oxygen atom, carboxyl group or carbonyloxy group. Favoured groups include straight chained alkylenyl groups —(CH₂)ₙ— where n is 1, 2, 3 or 4, and is preferably 2.

In conjunction with hydroxy substituted polymers, the spacer group X is aptly of the formula —CO—O—X1— or —CO—NH—X1— where X1 is a lower alkylenyl group. Suitable alkylenyl groups include —CH₂—CH₂—, —CH₂—CH₂—CH₂— and —CH₂—CH₂—CH₂—CH₂— groups which may optionally be interrupted with a hetero-atom, for example -O-, or substituted (for example by —OH). The —CO—NH—CH₂—CH₂— group is the preferred spacer group.

A particularly preferred value for the —X—Y—Z—R group for attachment to hydroxy group containing polymers is the —CO—NH—CH₂—CH₂—S—S—2-pyridyl group.

In addition to side chains of formula —X—Y—Z—R, the polymer may optionally also contain side chains of the formula —X—Y—Z—R1 where R1 is a member of a specific binding pair.

The term “specific binding pair” is used in respect of two molecules which have a specificity for each other so that under normal conditions they bind to each other in preference to binding to other molecules and most aptly to the essential exclusion of others. Suitable binding pairs include antibodies and antigens, ligands and receptors, and complementary nucleotide sequences. One or both members of the pair may be part of a larger molecule, for example the binding domain of the antibody, a section of a nucleotide sequence and the like. Ligands, for example hormones, cancer marker proteins and the like, may be suitable agents for binding to receptors on cell surfaces or the like to determine cells possessing said receptor. Suitable members of pairs may include molecular imprints, aptamers, lectins and the like. Typically the member of the specific binding pair coupled to the polymer will comprise a peptide or a polypeptide.

A particularly favoured member of a specific binding pair is an antibody. A fragment of antibody may be used as long as it possesses the binding fragment such as a Fd, Fv, Fab, F(ab prime)2 and single chain Fv molecules linked to form an antigen binding site.

One member of a specific binding pair may be attached to some of the side chain of chains on the polymer by replacing some of the —Z—R moieties by —Z—R1 moieties where R1 is the residue of the member of a specific binding pair. This may involve reaction with a thiol group naturally present in the member of the specific binding pair, for example a protein. Alternatively the HZ— group could be synthetically added to the member of the binding pair (in a manner which does not prevent the modified member binding with the other member of the pair). For example, hydroxy groups or amino groups could be acylated with an active derivative of mercaptopropionic acid or the like.

The polymer employed in this invention may be a synthetic or natural polymer. It may take the form of a hydrogel, a porous matrix, a gel, a crosslinked polymer, a star polymer, a dendrimer or the like. Such polymers may also be co- or ter- polymers, graft polymers, comb-polymers and the like.

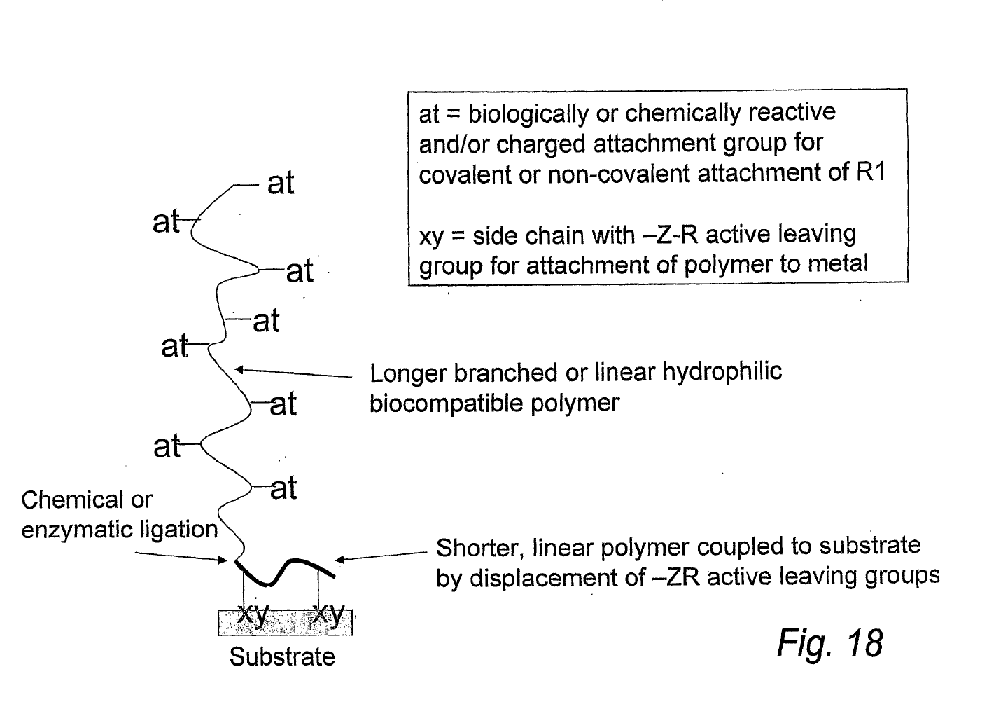

In particular, in one embodiment, the invention encompasses the use of complex polymer structures, in which the receptor moiety R1 is further removed from the surface to which the polymer is coupled. Such an embodiment is illustrated schematically in FIG. 18. In FIG. 18, a substrate is coated with a complex structure: a relatively short polymer in accordance with the invention is coupled to the substrate by displacement of —Z—R leaving groups from X—Y—Z—R side chains. A relatively long molecule or moiety is attached to the polymer (before or after coupling to the substrate), which relatively long moiety comprises a plurality of biologically or chemically reactive groups (“at”) for attachment of a receptor, antibody or other member of a specific binding pair. The inventors hypothesise that by placing the leaving groups —Z—R at a distal end of the polymer, thereby distancing the receptors R1 from the substrate, it may be possible to construct a biocompatible surface coating that is more permeable to both protein and small molecule binding, enabling higher receptor densities on the substrate and/or higher signal: noise ratios in biosensor applications. It may also be possible to reduce the effect of mass transport-limited binding relative to commercially available conventional polymer-coated surfaces in which the polymer is attached to the surface at multiple points, leading to a compact, less permeable matrix.

Referring again to FIG. 18, the relatively long moiety may be attached to the polymer by chemical or enzymatic ligation, or by polymer “grafting”. Thus a matrix may be formed from, for example, copolymers (including comb polymers, alternating copolymers, block copolymers) and terpolymers. Methods of polymerisation are well known to those skilled in the art and representative general teaching of suitable techniques is given by Matyjaszewski & Xia (2001, Chem. Rev. 101, 2921-2990) and Hawker & Wooley (2005 Science 309, 1200-1205).

It is preferable that the polymer used in this invention is hydrophilic, biocompatible and, when present as a coating, is able to resist non-specific binding of analytes and contaminants. Suitable polymers include dextran, hyaluronic acid, sepharose, agarose, nitrocellulose, polyvinylalcohol, partially hydrolysed polyvinylacetate or polymethylmethacrylate, carboxymethyl cellulose, carboxymethyl dextran and the like.

The use of the polymers of this invention can lead to an excellent level of coating of the metal which contributes to the reduction of non-specific binding observed in this invention. This is most easily achieved by using a neutral (uncharged) polymer. The term “neutral” as used herein, is intended to indicate that a polymer does not contain any readily ionisable groups, and therefore will be uncharged in all physiological environments.

Charged polymers according to this invention (for example those derived from polymers containing residual carboxylate groups such as carboxymethylated cellulose derived polymers) can be used to enhance binding of desirable moieties. For present purposes, a “charged” polymer is one which comprises readily ionisable groups, (e.g. —OH, —NH₂, —COOH). Accordingly, under certain conditions of pH or the like, a “charged” polymer may in fact be neutral, because the groups in question are uncharged or because the polymer comprises equal numbers of positive and negative charges, whilst in other conditions, the polymer will carry a net charge, due to ionisation of the readily ionisable groups.

It has been found that particularly good resistance to non-specific binding can occur with the use of neutral (uncharged) polymers of the invention.

Particularly apt derivatisable polymers for use in this invention are those derived from sugar monomeric units.

A preferred derivatisable polymer for use in this invention is dextran. Suitable grades of dextran include T10, T70 and T500.

Thus an alternative and generally more suitable method of producing polymer coatings containing —X—Y—Z—R1 groups is to react a polymer of this invention containing —X—Y—Z—R groups with a substrate, preferably a metal surface, so that at least some —Z—R groups are displaced and the polymer becomes attached to the metal surface via —X—Y— side chains, and thereafter reacting some or all of the remaining —X—Y—Z—R groups with a compound H—Z—R1 whereby the —Z—R groups become replaced by —Z—R1 groups.

The surface to be coated will preferably be a metal one that has reactivity with chalcogen containing molecules. Such metals include those of groups 10 and 11 and titanium. Favoured metals include gold, silver, platinum, palladium, nickel, chromium, titanium and copper and their alloys of which gold and platinum are most favoured. A preferred metal is gold.

Metal ions may also be employed such as cadmium, ferrous and mercurous ions. An adhesion coat may be present between the substrate and the metal if desired. A preferred adhesion coat comprises titanium.

The polymer of this invention possessing side chains containing —X—Y—Z—R groups, and optionally —X—Y—Z—R1 groups, can be reacted with a substrate, preferably a metal surface, so that at least some —Z—R groups are displaced and the polymer becomes attached to the metal surface via —X—Y— side chains.

Thus in one favoured aspect, this invention provides a metal surface coated with a hydrophilic polymer which has side chains of the formula —X—Y—Z—R1 wherein X, Y, Z and R1 are as previously defined. Apt, favoured and preferred values for X, Y and Z and combinations of X, Y and Z are as stated hereinbefore.

In the case where the polymer contains both of the preceding side chains, those with —Z—R groups will be displaced preferentially, thus providing an attached polymer which contains —X—Y—Z—R1 groups attached to the metal.

The metal surface is most suitably supported on a more robust substrate layer. This substrate may be of any convenient materials, typically (but not necessarily) suitable for use as a biosensor. In the case of conventional assay, plastics such as polystyrene are used, or glass. Piezoelectric or optical materials may also be used. Glass or quartz are the preferred substrates for optical biosensors, particularly surface plasmon resonance devices. Suitable piezo-electric materials are well known and include quartz, lithium tantalate, gallium arsenide, zinc oxide, polyvinylidene fluoride and the like, but quartz is most suitable.

The substrate may be employed in an active or a passive device for the detection and/or characterisation of a member of a specific binding pair which becomes bound to the other member of the specific binding pair which is present on the side chain attached to the polymer.

In an alternative method —ZR groups may be displaced by groups which contain a reactive moiety to which the member of the specific binding pair can become attached. In such an embodiment the group R1 can be considered as the residue of the specific binding pair which also incorporates a linker group at the point of attachment to the group Z.

Such an alternative method can be used for the attachment of the group R1 via an amino, hydroxy, carboxy or like group. For example, a polymer containing —X—Y—Z—R side chains can be reacted with N-succinimidyl 3-(2-pyridyldithio)propionate (hereinafter, SPDP) which then provides side chains —X—Y—S—CO—O-1-pyrrol-2,5-dione which on reaction with a primary amine within R1 provides a —X—Y—S—CH₂CONHR2 side chain wherein CH₂CONHR2 represents R1.

Additionally or alternatively, reactive groups of the polymer, which are not —Z—R groups, may be used to attach a member of the specific binding pair.

Such an alternative method can be used for the attachment of the group —R1 via an amino, hydroxy, carboxy, epoxy or like group, which is either present in the polymer before modification with the —X—Y—Z—R side chains, or which can be introduced to the polymer after introduction of X—Y—Z—R side chains. For example the hydroxy groups present in dextran can be modified after the polymer is immobilised on the surface via —X—Y—Z—R side chains, for example by reaction with bromoacetic acid under basic conditions, to create acidic carboxymethyl (—CH₂COOH) reactive groups. In a similar manner, the polymer can be reacted with tosyl chloride, mesyl chloride, cyanogen bromide, epi-bromohydrin, carbodiimides, bisoxiranes, divinyl sulphones, etc. to create neutral reactive groups. Such reagents for providing reactive groups are well known and understood by those skilled in the art. A table of suitable functional groups/activating reagents is provided in Appendix 1 annexed to the present description. The unreacted —Z—R groups may optionally first be inactivated (capped) by exposure to an aqueous solution of a Z-reactive capping reagent, (e.g. cysteine, in the case of Z=sulfur, in 100 mM sodium acetate buffer pH 4.5 with 1 M NaCl).

From the foregoing it will be appreciated that an aspect of this invention provides a metal coated with a polymer containing side chains of the formula —X—Y—Z—R1 wherein —X—, —Y—, —Z— and —R1 are as defined hereinbefore.

Preferably the polymer will be attached to the metal via side chains of the formula —X—Y—.

In this aspect apt, favoured and preferred values for —X—, —Y—, —Z— and —R1 are as defined hereinbefore as are the polymer and metal surface.

Similarly, it will be appreciated that in another aspect, this invention provides a substrate, at least one surface of which is coated with a metal, which metal is itself coated with a polymer containing side chains of the formula —X—Y—Z—R1 as defined hereinbefore. In a highly favoured aspect, this invention provided a biosensor which comprises a substrate and a metal coating on at least one face of the substrate, which metal coating is itself coated with a polymer containing side chains of the formula —X—Y—Z—R1 wherein —X—, —Y—, —Z— and —R1 are as hereinbefore defined.

The polymer will be attached to the metal via side chains of the formula —X—Y—. In this highly favoured aspect, apt, favoured and preferred values for —X—, —Y—, —Z— and —R1 as hereinbefore defined as are the polymer, metal surface and substrate. An adhesion coat may be present between the substrate and the metal if desired.

Such polymer coated metals may be used in a biosensor, for example when they coat a substrate. Hence the invention provides a substrate coated with a metal to which is attached a polymer by —X—Y—Z— moieties said polymer having —X—Y—Z—R and/or —X—Y—Z—R1 and/or derivatisable groups such as hydroxy, amino or carboxylic acid groups, or salts thereof wherein the metal, polymer and —X—, —Y—, —Z—, —R and —R1 groups are hereinbefore defined. An adhesion coat may be present between the metal and substrate if desired. The polymers of this invention containing —X—Y—Z—R side chains may be made in any convenient manner from the initial polymer. Thus for example polymers containing carboxyl groups may be esterified, polymers containing amino or hydroxyl groups may be acylated and other derivatisable groups may be derivatised using methods known to the skilled worker.

A particularly apt method of introducing the side chains into polymers containing hydroxy groups comprises first reacting the polymer with 4-nitrophenylchloroformate or analogous reagent in polar aprotic organic solvent preferably in the presence of an acid sequestering agent and a catalyst.

Suitable solvents include dimethylsulfoxide (DMSO) and solvents of similar properties. Suitable acid sequestering agents include amines such as pyridine. Typical catalysts include N,N-dimethylamino-4-pyridine. Generally the reaction is performed at ambient temperature with external cooling but any non-extreme temperature, for example 10-30° C., may be employed. The reaction may take 3 to 12 hours, for example 5 or 6 hours.

The degree of esterification of hydroxyl groups may be controlled by the amount of esterification agent, for example 4-nitrophenyl chloroformate, employed, the length of reaction time, temperature of reaction and so on in a manner that will be understood by the skilled worker.

Generally in the case of saccharide-based polymers, such as cellulose, dextran and their derivatives, aptly more than 3%, for example 3-60% of the available hydroxyl groups may be substituted, for example about 3-30%, more usually about 5-15%, for example 3, 7, 10, or 15%. (The percentage values represent the number of side chains per hundred sugar residues of the polymer). The use of good leaving groups and such levels of substitution enables greater polymer deposition than previously achieved by SAMs and allows for a correspondingly enhanced level of coupling of a member of a specific binding pair and hence eventual binding of analyte. Thus, for example, the polymer deposition achievable by the present invention is able to exceed 2.0 ng/mm², more aptly greater than 2.5 ng/nm², favourably greater than 3.0 ng/mm², more favourably greater than 3.5 ng/mm² and preferably greater than 5.0 ng/mm².

The acylated polymer may be recovered from solution by precipitation, for example by adding a miscible non-solvent such as a mixture of methanol and ether and then filtering off the precipitate.

The 4-nitrophenyl carbonated dextran may then be reacted with a compound of the formula NH₂—B—Y—Z—R to provide dextran with side chains of the formula —O—CO—NH—B—Y—Z—R wherein B, Y, Z and R are as hereinbefore defined and the —O—CO—NH— group represents the group of the formula A as hereinbefore discussed. The degree of derivatisation (number of side chains) reflects the degree of acylation of the intermediate carbonated polymer.

The reaction of the 4-nitrophenyl carbonated dextran and the amino compound will generally take place in a polar aprotic solvent such as dimethylsulfoxide at a non extreme temperature, for example ambient temperature. The reaction will generally take place in the presence of an acid acceptor such as pyridine and in the presence of a catalyst such as N-methyhnorpholine. The desired reaction product may be obtained by use of a miscible non-solvent such as a mixture of methanol and ether followed by filtration.

The previous two reactions may be performed consecutively without the isolation of the intermediate 4-nitrophenyl carbonated dextran if desired.

The apt, favoured and preferred values for the amine of the formula NH₂—Y—Z—R are as set out hereinbefore. Thus, for example a favoured amine for use is that of the formula H₂N—CH₂—CH₂−S—S—2Py.

If desired some of the active leaving groups may be displaced to yield side chains containing —X—Y—Z—R1 moieties as hereinbefore described. Alternatively, such side chains may be introduced after coupling of the polymer to a surface as described below.

In the manufacture of a biosensor it is well known to deposit a layer of metal on a substrate. Thus, for example a thin layer of titanium may be coated onto a substrate such as glass, quartz, plastic or the like. Such adhesion layers are generally formed by vapour deposition and are 0.5-5 nm thick, more usually 1-2.5 nm, for example about 1.5 nm thick. A thicker layer of a metal as hereinbefore described, particularly gold, platinum or silver and preferably gold, is then coated over the adhesion layer, for example by vapour deposition. The thickness of such metal layers depends on the biosensing technique to be employed and may be from about 10-200 nm, more usually about 20-100 nm, for example 35-75 nm thick. For piezoelectric methods the thickness is typically 100-200 nm for example.

The active side chain containing polymer may be bound to the surface of the metal by bringing a solution of the active side chain polymer into contact with the metal surface. Generally, before contacting the metal surface with the solution of the polymeric agent, the surface is cleaned, for example by washing with ultra-pure water, then with sodium hydroxide and surfactant solution and then more ultra-pure water.

The cleaned surface is then contacted with the solution of polymeric agent, for example for 3 to 30 minutes, more usually 10-20 minutes. Typically the solution may contain 0.1-10%, for example 0.5 to 7.5%, of polymer containing —X—Y—Z—R side chains. The contact may be static or the solution may be moved relative to the metal surface. During this time, —Z—R groups are displaced and the polymer becomes bound to the metal via —X—Y— groups. For example, in the case of a polymer containing —X—S—S—2Py groups, the —S—2Py group is displaced and the polymer attached to the metal by —X—S— bonds. After this binding has occurred, any non-chemisorbed polymeric material may be removed by washing with sodium hydroxide. The resulting polymer coated metal is unaffected by acid, base, salts, detergents or cysteine at concentrations likely to be encountered in use in a biosensor.

Since only some of the active leaving groups are displaced by binding to the metal, the metal surface is coated with a layer of polymer which polymer retains some —X—Y—Z—R side chains as hereinbefore described.

It is believed that the coating thus achieved covers a higher proportion of the metal surface than what has been achieved by prior art methods which have not employed good leaving groups —Z—R.

The —X—Y—Z—R group can be reduced to a free Y group, (—X—YH); for example where Y is sulfur, reduction to a sulfhydryl group (—SH); by a reducing agent, such as dithiothreitol (DTT), and then reacted with an agent such as SPDP or a sulphated analogue. The resulting polymer containing —X—Y—S—CH₂—CO—O—N═(COCH₂CH₂CO) side chains (or other N-hydroxysuccinamide analogues with a SO₄^{2—} salt) may then be reacted with amino groups present in R1 moieties or derivatised R1 moieties, for example proteins and particularly antibodies. If R1 is a small molecule (for example a ligand that binds to a receptor to be analysed) it may be linked by reaction with a sulphydryl or amino group it possesses or it may be derivatised to include such a group. Thus, for example a hydroxy group may be esterified with 3-mercaptopropionic acid or glycine or the like. Similarly, a carboxy group may be esterified with NH₂CHCH₂OH, HSCH₂CH₂OH or the like. Alternatively, a cross reactive analogue of a natural ligand can be used which contains a sulphydryl or amino group or a derivatised hydroxy or carboxy group or the like.

In an alternative process the metal surface may be contacted with a polymer containing both —X—Y—Z—R and —X—Y—Z—R1 side chains. The polymer becomes bound to the metal surface by displacement of —Z—R groups leaving —X—Y—Z—R1 side chains in place, since —Z— R is a better leaving group than —Z—R1.

In a further alternative process the metal surface may be contacted with a polymer containing —X—Y—Z—R side chains. The polymer becomes bound to the metal surface by displacement of —Z—R groups, and other groups present in the polymer before modification are converted to reactive side chains.

Any residual active disulphide groups may be inactivated (capped) by exposure to an aqueous solution of cysteine, for example in 100 mM borate buffer at pH 8.5.

It will be appreciated that, in a favoured aspect, this invention provides a biosensor comprising (i) a substrate; (ii) a layer of metal on a surface of said substrate; (iii) a polymer attached to said metal by side chains of the formula —X—Y—; and (iv) said polymer also having side chains of the formula —X—Y—Z—R1; wherein X, Y, Z and R1 are as hereinbefore defined.

Preferably, but not necessarily, the X—Y groups in the two types of side chain are the same. The groups X, Y, Z and R1 are aptly, favourably and preferably as hereinbefore described. The substrate and metal will aptly, favourably and preferably be as hereinbefore described. The polymer will aptly, favourably and preferably be as hereinbefore described.

In one embodiment the polymer may be derivatised with lipophilic groups or reactive species that can bind non-covalently or covalently to lipids, vesicles, liposomes, membrane fragments, cells, enveloped viruses or other lipidic entities.

Supported lipid bilayers and monolayers have been widely utilised in combination with acoustic and optical biosensors to analyse interactions with many varied membrane-receptor-ligand systems. In general, these assemblies are based on gold or silver films that, due to the evaporation or sputtering process of deposition, possess intrinsic surface roughness on the molecular scale. The monolayer and bilayer assemblies that are closely coupled to these metallic surfaces can be polycrystalline or amorphous in nature, and hence do not fully mimic a fluid membrane. In addition, the surface roughness of most of the materials used as a base for the bilayer (SiO₂, glass, metals, polymers, etc.) prevents the undisturbed organisation of lipids and/or native membranes as a mono-molecular layer (Duschl & Knoll 1988 Journal of Chemical Physics 88, 4062-4069; Spinke et al, 1992 Biophysical J. 63, 1667-1671).

In order to overcome these problems, pioneering work was carried out by the groups of Sackmann, Ringsdorf and Knoll, who investigated the formation of lipid bilayers bound to, but structurally decoupled from, the solid support by a flexible polymer. These soft polymer cushions provide a lubricating layer between the surface and the membrane, and enable the “self-healing” of surface defects that increase the degree of non-specific binding to the surface. Three different basic strategies have been employed: a) the chemical grafting to the solid surface of a ultra-thin film of a water-soluble natural polymer such as dextran or hyaluronic acid, which has been derivatised with long alkyl chains that can insert into, and anchor membranes (e.g. Cooper et al, 2000 Anal. Biochem. 277, 196-205; Jenco et al, 1997 Biochemistry 327, 431-437), b) coupling to the surface lipopolymers which possess functionalised head groups (e.g. Sackmann 1996 Science 271, 43-48) and, c) the deposition of soft hydrophilic multilayers of rod-like molecules with alkyl side chains which insert into and anchor membranes (e.g. Wiegand et al, 1997 J. Colloid Interface Sci. 196, 299-312; Erdelen et al, 1994 Langmuir 10, 1246-1250; and Beyer et al Angewante Chemie 35, 1682-1685).

The reader is also referred to US 5,922,594 (especially column 3 thereof) and WO 02/072873 (especially page 7 thereof) for further description of methods of immobilising lipid structures to solid supports.

Thus polymers in accordance with the invention might be useful in immobilising lipophilic groups on surfaces, which could act as artificial or pseudomembranes to study, for example, the behaviour of vesicles, liposomes or membrane-bound receptors or other membrane-bound molecules.

As mentioned previously, the present invention provides a biosensor comprising the polymer defined hereinbefore.

The biosensor may be seen as a combination of a receptor for molecular recognition (the immobilised R1 groups bound to the polymer which is bound to the substrate via the metal) and a transducer for transmitting the interaction as processable signals. Examples of optical biosensors may be seen in US2002/012577 pages 2 and 3 of which are incorporated herein by cross reference.

Suitable biosensors include optical biosensors, for example those employing surface plasmon resonance, attenuated total internal reflection, FTIR, resonant colorimetric reflection, resonant mirror, fluorescence, luminescence, chemiluminescence or the like.

Other suitable biosensors include acoustic biosensors, for example quartz crystal mass sensors, such as transverse shear wave or surface acoustic wave devices. In such biosensors the polymer layer performs the additional function of transmitting an acoustic wave to and from an acoustic wave device to and from an immobilised group R1 in order to sense interactions with the other member of the specific binding pair (the analyte).

Equally, other suitable biosensors include those determining electrical properties, such as conductimetric and dielectric sensors, for example those employing field effect transistors.

A further class of biosensors are force biosensors, for example atomic force microscope, biophase membrane probe and the like, a microelectromechanic sensor, calorimetric, dielectric, conductimetric biosensors and microtitre plates.

In the case of force biosensors or micromechanical biosensors, the polymer performs the additional function of transmitting a motion or applied force to and from the force sensor or, micromechanical sensor, to and from the immobilised group R1 in order to sense interactions with the other member of the specific binding pair (analyte)

If desired biosensors may be in the form of microassays in which the polymer is immobilised over each of the assays.

A preferred biosensor of this invention is a surface plasmon resonance biosensor.

A favoured form of this aspect of the invention provides an optical biosensor, and a particularly favoured form of optical biosensor is a surface plasmon resonance (SPR) biosensor, comprising (i) a substrate, preferably a glass substrate, (ii) a layer of gold, silver or platinum, preferably a layer of gold, (iii) a hydrophilic polymer attached to the gold, silver or platinum by S—X moieties; and (iv) said polymer having side chains of the formula —X—S—S—R1 wherein X and Ri are as hereinbefore defined.

A particularly favoured form of this aspect of the invention provides a biosensor comprising (i) a piezoelectric substrate, preferably a quartz substrate, (ii) a layer of gold, silver or platinum, preferably a layer of gold, (iii) a hydrophilic polymer attached to the gold, silver or platinum by S—X moieties; and (iv) said polymer having side chains of the formula —X—S—S—R1 wherein X and R1 are as hereinbefore defined.

Another particularly suitable biosensor of this invention is an acoustic biosensor.

In use, the solution to be analysed may be derived from a biological or other source. For example, a bodily fluid, cell extract, food material, scientific sample or the like. Such solutions may include as analyte a chemical, drug, steroid, tissue, membrane, membrane fragment, nucleotide, oligonucleotide, protein, oligosaccharide, cell, phage, bacteria, virus or any other structure which contains groups capable with specific interactions with another member of a specific binding pair.

In use the analyte may be present in a crude preparation or in a partially purified preparation. The analyte solution may be diluted with a buffer solution if desired and may contain salts if required. Thus, for example a source suspected of containing the analyte may be diluted with phosphate buffered saline containing NaCl and/or KCl at pH 7.4.

The analyte solution may be passed over or otherwise contacted with the biosensor to which the binding partner of the analyte has been immobilised. The analyte binds to its binding partner and the sensor measures the binding. After the measurement is complete the binding partner may be regenerated by washing with e.g. water and/or phosphate buffer until dissociation has allowed the analyte to be removed.

Such analysis generally takes place at a non-extreme temperature, for example ambient temperature.

Thus, this invention provides a method of analysing for a member of a specific binding pair which comprises contacting a sample suspected on containing that member of a specific binding pair with a biosensor of the invention in which R1 is the other member of the specific binding pair and noting the change in signal from the biosensor.

Without wishing to be bound by any particular theory, it is believed as a working hypothesis that the use of uncharged hydrophilic polymers in this invention result in a layer over the metal surface which is believed to be more complete than that achieved in known systems, for example those in which highly charged polymers such as carboxymethyl cellulose or carboxymethyl dextran have been employed. Such charged polymers appear to lead to thick, highly swollen layers, which are however sufficiently porous to allow penetration of non-specific binding materials to areas of the metal surface and which leads to high backgrounds or false signals. Use of uncharged hydrophilic polymers provide layers which are not highly swollen and cover the surface to a degree of completeness that reduces and can effectively eliminate non-specific binding so that high backgrounds and false signals are greatly reduced or eliminated.

The invention may however also be used as a means of attaching polymers which can have charged groups of the type described in Reference 12. The charged groups have the function of pre-concentrating ligands when the conditions of attachment such as the pH are adjusted to provide a charge on the ligand which is opposite to that of the polymer. Suitable X—Y—Z—R groups of the invention may be incorporated (as described herein) into the polymer, and following immobilisation of the polymer on the metal the conditions adjusted to provide the preconcentration conditions. The reactive groups present in the charged polymer may comprise X—Y—Z—R1 groups that are derived from residual X—Y—Z—R groups, X—Y—Z—R1 groups already attached to the polymer, or may be generated from other groups present in the polymer before modification.

Suitable intermediates for coupling to the polymers to provide the side chains can be made by methods known in the art. References 32-40 may be consulted for suitable methods of providing intermediates. Where the polymer is a hydroxylic polymer it may be converted to a p-nitrophenyl carbonate derivative as hereinbefore described and as illustrated in the Examples hereinafter. Such a p-nitrophenyl carbonate derivatised polymer may then be reacted with an amine NH₂—B—Y—Z—R where B, Y, Z and R are as hereinbefore defined.

Such reactions are generally carried out at ambient temperature or with optional cooling. Frequently a non-hydroxylic but hydrophilic solvent such as DMSO will be employed.

The amine NH₂—B—Y—Z—R may be prepared by reaction of NH₂—B—Y—H with a compound R—Z—Z—R or the like. Such reactions are particularly apt when Z is S.

The preparation of mixed thiosulfonates (RSO₂SR3) may be made in known manner. For example an alkyl halide such as R3-halogen, when R3 is alkyl, may be used to alkylate RSO₂SNa or RSO₂SK; equally this is possible from an aryl halide such as R3-halogen when R3 is aryl, it is possible to prepare the sulfenyl halide and use it to derivitise RSO₂SNa, RSO₂SZn or the like. Trifluoromethyl thiosulfonates may be used to derivatise a compound containing a SH group by direct reaction.

Various amino-substituted thiosulfonates are known (for example see Ref. 32 and Ref. 33). A general method employs a thiol, such as cysteine hydrochloride, which is reacted with trifluoromethyl p-toluenethiolsulfonate in a solvent such as ethanol, with stirring at ambient temperature to yield methane thiosulfonic acid S-(2-amino-ethyl)ester. This compound was also described in Ref. 33. Use of this compound to react with p-nitrophenyl carbonate dextran would provide the compound containing —CO—O—CH₂—CH₂—S—SO₂—CF₃ side chains.

It is possible to obtain the required intermediates for any chalcogenide by methods in the art such as Refs. 35-39. Thus for example a selenanyl-, sulfyl-, telluryl-chloride (1 mmol), m-CPBA (5 mmol) and 10% potassium hydroxide (2 ml) in isopropyl alcohol (20 ml) may be stirred at room temperature for 1 h. The solution may be diluted with saturated sodium thiosulfate (10 ml) and extracted with chloroform (2×20 ml). The combined organic extracts maybe washed with 10% sodium hydroxide (10 ml), dried, the solvent removed under reduced pressure and the residue purified by chromatography.

These functional intermediates can then be converted into required amino-ester by reaction under acidic conditions with an aminoalkyl chalcogenide such as cystamine. Alternatively, this procedure could be carried out in two steps in an aprotic solvent, with a suitably protected amino group such as the Fmoc, or Alloc group.

For the avoidance of doubt, it is hereby expressly stated that features of the invention described herein as apt, suitable, favoured, preferred or the like may be employed in the invention in isolation or in any combination with any other features so described, unless the context dictates otherwise.

The invention will now be further described by way of illustrative example and with reference to the accompanying drawings, in which:

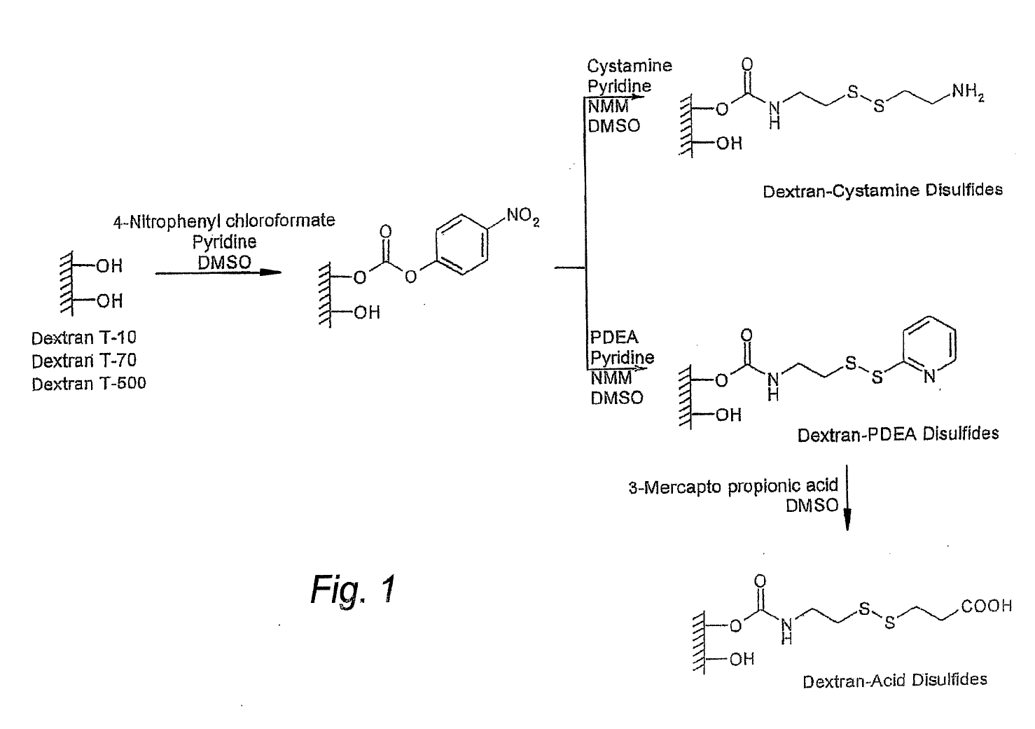

FIG. 1 is a schematic illustration of the synthesis of active leaving group polymers, some of which are in accordance with the present invention;

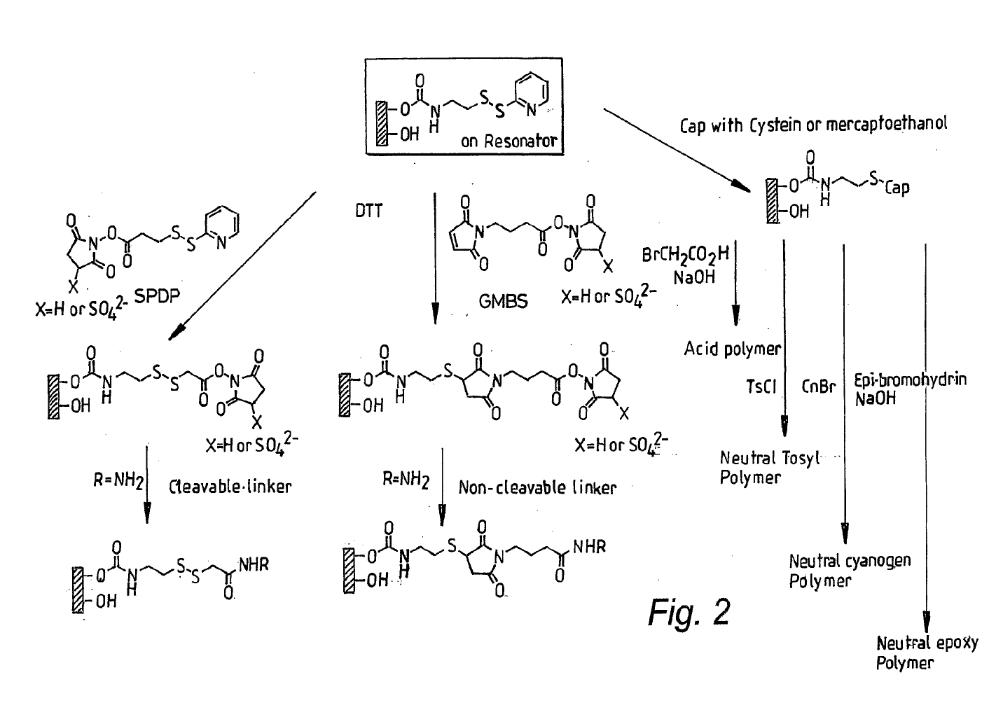

FIG. 2 is a schematic illustration of the introduction of members of specific binding pairs into polymers in accordance with the invention;

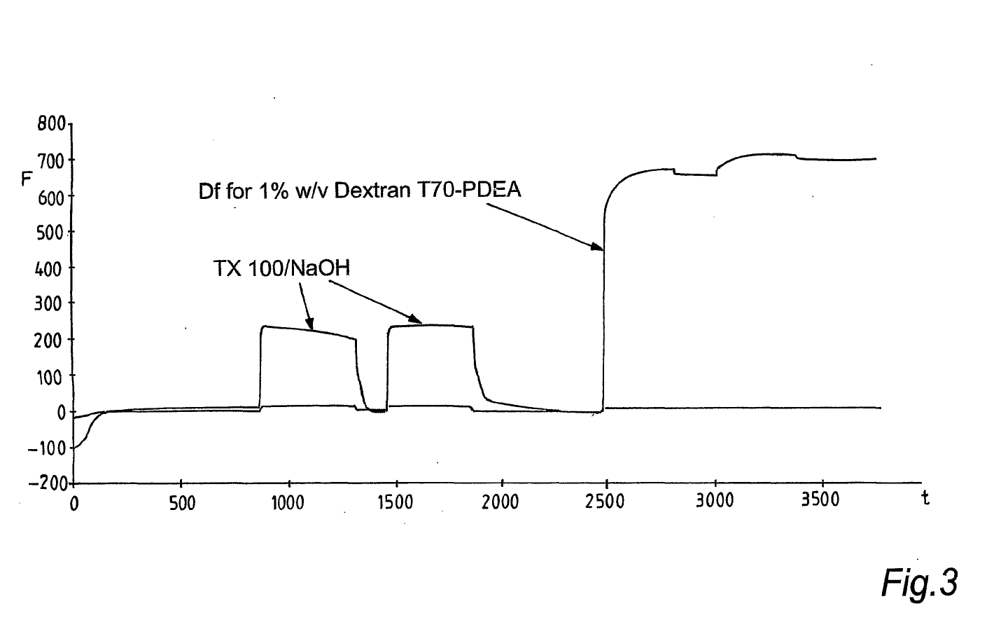

FIGS. 3-5 and 7-17 show representative results obtained in experiments described in the examples below;

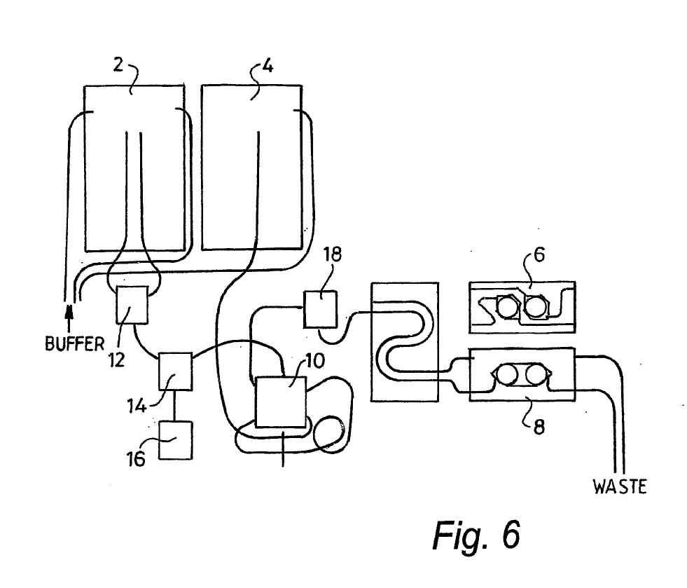

FIG. 6 is a schematic illustration of quartz crystal resonance sensing apparatus used in the examples described below; and

FIG. 18 is a schematic representation of one embodiment of a polymer-coated surface in accordance with the invention.

### EXAMPLES

In the following examples those in relation to cystamine disulfide and to acid disulfides are for comparison purposes only.

The synthesis of active leaving group polymers is illustrated schematically in FIG. 1.

Intermediate 1

Synthesis of (2-(pyridinyldithio)ethaneamine (PDEA)

Alrithiol-2 (4.41 g, 20 mmol) was dissolved in 20 ml of methanol and 0.8 ml of acetic acid. Into the solution was added 2-aminoethanethiol hydrochloride (1.14 g, 10 mmol) in 10 ml of methanol. The reaction mixture was stirred for 2 days, then concentrated in vacuo to give a yellow oil. The yellow oil was then thoroughly washed by vigorously stirring with diethyl ether. The clear yellow supernatant was decanted off, and the residue dissolved in methanol (10 ml). To the resultant solution was added 50 ml of ether, and the precipitate separated by filtration. This procedure was repeated three times, then the resultant final solid purified by recrystallisation (methanol/diethyl ether) to give a pale white solid (1.62 g).

Intermediate 2

4-Nitrophenyl Carbonated Dextran (Ref. No.: AKU34-103):

To a solution of dextran T70 (1.6 grams, 29.6 mmol OH) in 18 ml of anhydrous DMSO and 16 ml of anhydrous pyridine, were added 4-nitrophenyl chloroformate (800 mg, 4 mmol) and catalytic amounts of DMAP (N, N-Dimethylamino-4-pyridine) with stirring at 0° C. (external cooling bath). The reaction mixture was stirred at this temperature for 1 hour, then at room temperature for a further hour. The solution was slowly added into a mixture of methanol and ether (1:1) (150 ml) with vigorous stirring. The precipitates formed were collected with filtration, and washed with the same solvent mixture 3 times. The collected white solid was dried under high vacuum to give 1.54 grams of white powder. 4-Nitrophenol (14.3 mg) and 4-nitrophenyl carbonated dextran AKU34-103 (22.3 mg) were dissolved in d⁶-DMSO (1 ml). ¹H NMR (400 MHz, d⁶-DMSO) (v52372): δ_{H}6.90(2H of 4-nitrophenol, δ_{H}, 7.54(2H of AKU34-103, δ_{H}, 8.08(2H of 4-nitrophenol, δ_{H}, 8.30(2H of AKU34-103, Functionality degree: 6.6% (mol, glucose unit).

When the reaction mixture was stirred at different temperatures, or the concentration of reactants was altered, different degrees of derivatisation were achieved as follows:

TABLE 1

| | Mol ratio of 4- | Mol % |
|---|---|---|
| Incubation with 4- | Nitrophenylchloroformate/ | derivatisation/ |
| Nitrophenylchloroformate | Dextran | glucose |
| 2 h @ 0° c. | 0.07 | 1.6 |
| 5 h @ 0° C. | 0.10 | 3.0 |
| 1 h @ 0° C. then 1 h at RT | 0.14 | 6.6 |
| 5 h @ 0° C. then 1 h at RT | 0.14 | 14.9 |

### Comparative Example 1

Dextran-Cystamine Disulfide (Ref. No.: AKU34-107):

To a solution of 4-nitrophenyl carbonated dextran AKU34-103 (750 mg) in 12 ml of anhydrous DMSO and 3 ml of anhydrous pyridine, were added NMM (N-methyl morpholine) (200 1) and cystamine dihydrochloride (1 gram, 8.8 mmol). The mixture was stirred at room temperature overnight when it was slowly added into a mixture of methanol and ether (1:1) (75 ml) with vigorous stirring. The precipitates formed were collected with filtration, and washed with the same mixture 3 times. The collected white solid was further dried under high vacuum to give 530 mg of white powder. Functionality degree; 6.6%

### Comparative Example 2

Preparation of Dextran-Acid Disulfide: (Dextran T70)

Dextran-Acid Disulfide (Ref. No.: AKU34-118):

To a solution of Dextran-PDEA disulfide AKU34-110 (500 mg) in 12 ml of anhydrous DMSO, was added 3-mercapto propionic acid (200 μl, 2.3 mmol). The mixture was stirred at room temperature overnight when it was slowly added into a mixture of methanol and ether (1:1) (50 ml) with vigorous stirring. The precipitates formed were collected with filtration, and washed with the same mixture 3 times. The collected white solid was dried under high vacuum to give 450 mg of white powder. Functionality degree 10.5%

### Example 1

Preparation of Dextran-PDEA Disulfide: (Dextran T70)

4-Nitrophenyl Carbonated Dextran (Ref. No.: AKU34-106):

To a solution of dextran T70 (1.6 grams, 29.6 mmol OH) in 18 ml of anhydrous DMSO and 16 ml of anhydrous pyridine, were added 4-nitrophenyl chloroformate (800 mg, 4 mmol) and a catalytic amount of DMAP with stirring and external cooling bath (0° C.). The reaction mixture was stirred at this temperature for 5 hours. The solution was slowly added into a mixture of methanol and ether (1:1) (150 ml) with vigorous stirring. The precipitates formed were collected with filtration, and washed with the same solvent mixture 3 times. The collected white solid was dried under high vacuum to give 1.57 grams of white powder.

4-Nitrophenol (11.6 mg) and 4-nitrophenyl carbonated dextran AKU34-106 (25.9 mg) were dissolved in d⁶-DMSO (1 ml). ¹H NMR (400 MHz, d⁶-DMSO) (v52603): δ_{H} 6.90(2H of 4-nitrophenol, d), 7.54(2H of AKU34-106, d), 8.08(2H of 4-nitrophenol, d), 8.30(2H of AKU34-106, d). Functionality degree: 10.5% (mol, glucose unit).

The synthesis was twice repeated with very similar results. The product of the second synthesis was referred to as AKU 34-175, and the product of the third synthesis was referred to as No. 110105.

Dextran-PDEA Disulfide (Ref. No.: AKU34-110):

To a solution of 4-nitrophenyl carbonated dextran AKU34-106 (1.57 grams) in 20 ml of anhydrous DMSO and 6 ml of anhydrous pyridine, were added NMM (400 μl) and PDEA (2-(Pyridin-2-yldisulfanyl)-ethylamine) (640 mg). The mixture was stirred at room temperature overnight when it was slowly added into a mixture of methanol and ether (1:1) (150 ml) with vigorous stirring. The precipitates formed were collected with filtration, and washed with the same mixture 3 times. The collected white solid was dried under high vacuum to give 1 gram of white powder.

¹H NMR (400 MHz, d⁶-DMSO) (v52718): δ_{H} 7.21 (1H, t), 7.42 (1H, br), 7.75 (1H, d), 7.81 (1H, t), 8.43 (1H, d). Functionality degree; 10.5%.

This synthesis was repeated under similar conditions and similar results obtained. The product of the second synthesis was identified as AKU34-178.

Preparation of Dextran-COOH-PDEA Disulfide: (Dextran T70)

Carboxymethyl Dextran (428015)

1.6 grams of dextran T70 (about 10 mmol glucose unit) was dissolved in 4M NaOH aqueous solution (25 ml) (4 g NaOH in 25 ml water). To this, was added a solution of MCA (monochloroacetic acid, 7.56 g) and Na₂CO₃ (4.24 g) in 20 ml water. The resulting solution was stirred at 90-100° C. for 3 hours. Then it was acidified with concentrated HCl aqueous solution with a cooling bath of ice-water to pH3.0, and purified by dialysis against distilled water until pH7.0 was obtained, and dried by lyophilization to give a white fluffy solid.

Functionality Degree=0.64 (Functionality degree is the % of substituted COOH of total OH in unsubstituted glucose) IR: 1733.7 cm⁻¹, 1635.4 cm⁻¹.

Dextran-COOH -PDEA (428018)

500 mg of Dextran T70-COOH (428015, D.S. 0.64) was dissolved in 16 ml of anhydrous DMSO at 60° C. The resulting clear solution was then cooled to room temperature, and to it, were added 383 mg of HOBt (2.5 mmol) and 0.78 ml of diisopropyl carbodiimide (DIC). The mixture was stirred at room temperature for 10 minutes, and then 3 ml of pyridine and 0.55 grams of PDEA was added. The resulting mixture was stirred at room temperature overnight. It was then dropped into 50 ml of acetone. To the resulting mixture were added 20 ml of ether and 200 ml of petroleum ether. The resulting precipitates were then collected by filtration, further washed with acetone 5 times, and dried under high vacuum to give a yellow powder 508.3 mg.

NMR data for the pyridinyl group/dextran were obtained as follows: ¹H NMR (300 MHz, D₂O) δ_{H} 8.41 (1H, br), 7.81 (2H, br), 7.30 (1H, br). Microanalysis (%): N=3.7 and 3.7, functionality: 1.32 mmol/g.

Preparation of Dextran-thiosulphone (Dextran-T70)

Sodium methanethiosulfonate (Ref. No. 428028):

A mixture of sodium methanesulfonate (1 gram, 9.8 mmol) and sulfur (312 mg, 9.75 mmol) in methanol (60 ml) was heated under reflux for 3 hours by which point all sulfur had dissolved. The mixture was then filtered, and the filtrate was concentrated in vacuo to dry to give a white solid.

¹H NMR (300 MHz, D₂O) _{H}3.375(3H, s)

IR: 1322.9 cm⁻¹, 1201.5 cm⁻¹, 1097.3 cm⁻¹, 977.7 cm⁻¹, 769.3 cm⁻¹

S-(2-Aminoethyl) methanethiosulfonate hydrobromide (Ref. No. 428031):

2 grams of sodium methanethiosulfonate (428028) (15 mmol) and 2 grams of 2-bromoethylamine hydrobromide (10 mmol) were dissolved in 50 ml of methanol. The resulting solution was then refluxed for 3 hours, and it was concentrated in vacuo to dry. The residue was suspended in 30 ml of acetonitrile, and the precipitates were filtered off. The filtrate was concentrated in vacuo to form a yellow sticky oil. To the residue, was added a mixture of acetonitrile and diethylether (2.5 ml / 2.5 ml) and the mixture was stirred vigorously overnight to give a yellow pasty solid. The remaining liquid was poured off and the solid was suspended in above solvent mixture (2.5 ml/2.5 ml) and triturated. The mixture was filtered, and the solid was washed with the above solvent mixture, and ether to give an off-white solid, and dried in vacuum with P₂O₅ to give 1.45 gram powder.

¹H NMR (300 MHz, D₂O) _{H}3.497 (5H, m), 3.371 (2H, t, J=17), 4.240 (2H, br) ¹³C NMR (300 MHz, D₂O) _{C}50.994 (CH₃), 41.016 (CH₂), 34.149 (CH₂) IR: 1309.4 cm⁻¹, 1132.0 cm⁻¹

Methanethiosulfonated Dextran T70 (Ref. No. 428034):

600 mg of 4-nitrophenylcarbonated dextran T70 (110105, 13.25%) was dissolved in 12 ml of DMSO and 3 ml pyridine. To this solution were added 200 μl of NMM and 300 mg of S-(2-aminoethyl) methanethiosulfonate hydrobromide. The resulting solution, was stirred at room temperature overnight, and was precipitated in a solvent mixture of methanol and ether (1:1) (75 ml) and the precipitates was collected by filtration and washed with the same solvent 3 times, and dried under high vacuum to give a white powder.

Microanalysis (%): N=0.66 and 0.79, S=4.00 and 4.10

Functionality: 0.58 mmol/g

Storage of and Preparation of Polymers

All polymers were stored as lyophilised powders at −20° C. under nitrogen. Between 0.1 and 2% w/v solutions of polymer in ultra-pure water were prepared by gentle shaking at room temperature until the solution was visibly clear, then they were centrifuged at 14000 rpm in a Genofuge 16M bench-top centrifuge to precipitate any undissolved material. The supernatant was then carefully removed with a pipette and stored at 4° C. until use.

### Example 2

Proteins and Reagents

Anti-HSA and anti-BSA mouse monoclonal antibodies (AbCam, UK) were stored at 1 mg/ml (ca. 1.5 μM) at 4° C. then diluted in running buffer to 333 nM for subsequent binding assays. Protein concentration was determined by the method of Bradford using a Bio-Rad protein assay dye reagent. Protein purity was determined by SDS-PAGE. Triton X-100, bovine serum albumin, human serum albumin, cysteine, glycine, dithiothreitol, sodium chloride, sodium hydroxide, hydrochloric acid, coupling buffers (10 mM sodium acetate buffer pH 4.5, 100 mM formate buffer, pH 4.3 and 100 mM borate buffer pH 8.5) were purchased from Sigma-Aldrich, UK and relevant solutions thereof filtered through a 0.22 μm filter before use.

Fabrication of Gold Surfaces

A number 2 Corning glass slide was coated with a titanium adhesion layer, then a 47 nm layer of gold in a Showa e-beam evaporator. The glass slide was mounted on a plastic holder suitable for insertion into a BIACORE®™ 2000 surface plasmon resonance (SPR) biosensor (Biacore, UK). SPR glass chip blanks were fabricated using AF 45, 0.30 nm thick glass slides (Perfection, Camb. UK) coated by vapour deposition with a 1.5 nm titanium adhesion layer and a 47 nm gold layer in an e-beam evaporator (Showa). The fabricated sensor chip formed four flow cells of dimensions 2.4×0.5×0.05 mm (1×w×h) in the instrument with a probing spot for the SPR signal of ca. 0.26 mm² for each flow cell. All SPR experiments were carried out at 25° C. with data points taken every 0.5 s.

Deposition of Polymers—SPR

The BIACORE®™ 2000 biosensor system was primed with ultra-pure water, then the surface of the gold chip cleaned by an injection of 40 μl of a solution of 100 mM NaOH/1% TritonX-100 at a flow rate of 10 l/min. In between each injection, running eluent was ultra-pure water. Immediately following this injection, 50 μl of a solution of an active leaving group polymer was injected at 5 μl/min. This resulted in a stable response level under continuing flow of water up to the maximum flow rate obtainable in the instrument (100 μl/min). Typical results are illustrated in FIG. 3, which is a graph of change in frequency (dF, Hz) against time (in seconds). Non-chemisorbed material could be removed with a pulse of 100 mM NaOH, resulting in a very stable SPR signal that was unaffected by further injections of any of 100 mM NaOH, 100 mM HCl, 1 M NaCl, 1% Triton X-100, 1 mM DTT, and 1 mM L-cysteine. For PDEA-polymers the response was in the order of ˜3500 RU (corresponding to ca. 3.5 ng/mm²) of polymer bound to the surface, and for thiosulphone polymers (Examples 11-13 below) the response was in the order of ˜2200 RU (corresponding to ca. 2.2 ng/mm²) of polymer bound to the surface.

### Example 3

Coupling of Proteins to PDEA-polymers—SPR

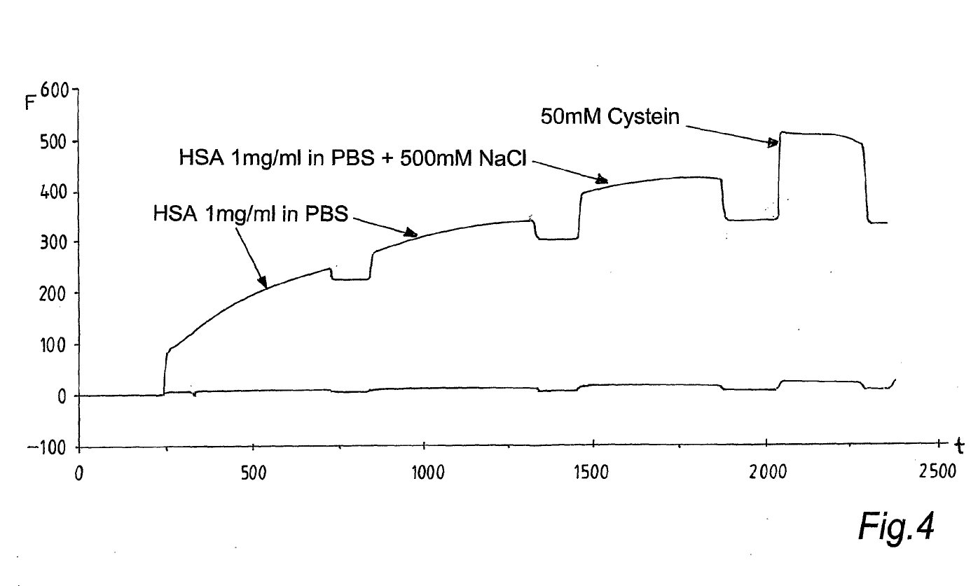

Direct coupling to protein sulhydryl groups: A solution of human serum albumin made up in 100 mM borate buffer/i M NaCl, pH 8.5 (50 μl, 1 mg/ml) was injected over the polymer-decorated surface, resulting in the immobilization of 1000-2000 RU of protein. Residual active disulfide groups were then inactivated (capped) by an injection of 50 mM cysteine in 100 mM borate buffer pH 8.5 (50 μl). Typical results are illustrated in FIG. 4, which is a graph of dF (Hz) against time, in seconds.

In a similar manner a control protein, bovine serum albumin (BSA) was immobilised on a different flow cell in the BIACORE®™ 2000 biosensor.

Amine coupling via SPDP:
1. Reduce with DTT
2. Activate with SPDP
3. Couple protein at pH 7.0
4. Cap with ethanolamine

### Example 4

Coupling of proteins to commercial BIACORE®™ CM5 surfaces—SPR

Equal volumes of NHS (50 μl, 50 mM in water) and EDC (50 μl, 200 mM in water) were mixed together, then 50 μl of this solution injected at 10 μl/min across a BIACORE®™ CM5 carboxymethyldextran sensor chip. This was followed immediately by an injection of HSA in 10 mM NaOAc buffer, pH 4.5 (50 μl, 50 μg/ml), resulting in the immobilization of ˜8000 RU of protein. Residual NHS esters were then inactivated by an injection of ethanolamine (50 μl, 1 M, pH 8.0).

### Example 5

Binding of Antibodies to Polymer-captured Protein Receptors—SPR

Mouse anti-HSA IgG was diluted three-fold in running buffer (PBS: 10 mM Na₂HPO₄/NaH₂PO₄, 137 mM NaCl, 2.7 mM KCl, pH 7.4) from 333 to 4.1 nM and then passed serially at a flow rate of 20 μl/min over a flow cell containing immobilised BSA, then over a flow cell containing immobilised HSA. Kinetic assays were performed with a 5 min. injection of IgG and with ca. 1500 RU of immobilized albumin. The sample solution was then replaced by running buffer, and the antibody-ligand complex allowed to dissociate for 5 minutes. Regeneration of the free protein receptor was effected by injection of a solution of salt (5 μl, 10 mM NaCl, pH 2.0).

Comparative experiments on a commercial BIACORE®™ CM5 sensor chip were carried out as described above with antibodies passed first over an underivatised flow cell, then over a flow cell with immobilised BSA, then over a flow cell with immobilised HSA. All assays were carried out at 25° C.

Non-specific binding properties of the polymer surfaces were assayed by injections of a non-relevant analyte or matrix. This was typically an anti-BSA monoclonal antibody (100 μl, 50 μg/min in PBS, 20 μl/min), a rabbit-anti-mouse polyclonal antibody (100 μl, 50 μg/min in PBS, 20 μl/min) or whole (undiluted) human serum (100 μl, 20 μl/min).

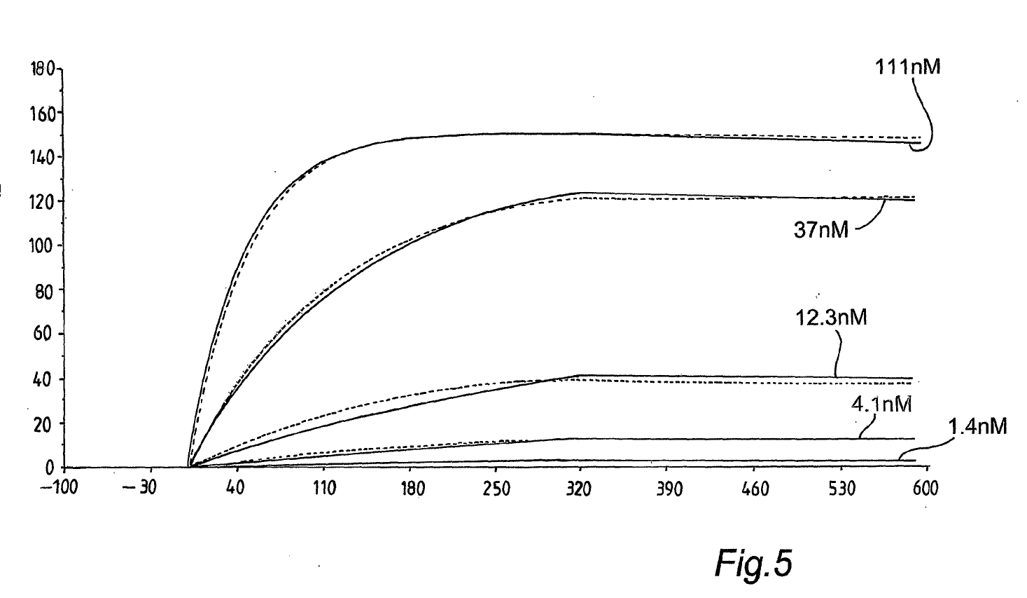

Changes in the SPR angle, given in response units, are proportional to the amount of material in the immediate vicinity of the sensor chip surface. As solutions of an analyte are passed over the surface, the affinity and kinetics of the binding event can be calculated from analysis of the resultant binding curve. Typical results are shown in FIG. 5, which is a graph of response (RU) against time (seconds). SPR data were prepared for analysis by subtracting the average response recorded 20 s prior to injection and adjusting the time of each injection to zero. Data from the flow cell containing BSA alone was subtracted from corresponding data obtained from the HSA-containing flow cell to correct for bulk refractive index changes and the effects of drift. Analysis was carried out using BIAeval 3.0 global analysis software based on algorithms for numerical integration. For the simple bimolecular model, A+B=AB, the process was assumed to be pseudo first order with no interaction between separate receptor molecules. The results are summarised in Table 2 below. In the Table, R max is a fitted parameter corresponding to the maximum signal that would occur if the analyte was present in substantial excess. As the actual experiments were not conducted under these conditions the Rmax value in Table 2 is not attained by the plots shown in FIG. 5. Instead the plots approach Req, which is a function of the analyte concentration.

TABLE 2

| | | | Rmax | Conc | | KA | | Req | kobs | |
|---|---|---|---|---|---|---|---|---|---|---|
| | ka (1/Ms) | kd (1s) | (RU) | of analyte | | (1/M) | KD (M) | (RU) | (1/s) | Chi2 |
| Anti-HSA 111 nM- | | | 152 | 11 | nM | | | 151 | 0.0217 | |
| HSA-dF (Hz) | | | | | | | | | | |
| 37 nM Anti-HSA- | | | 139 | 37 | nM | | | 137 | 7.30E−03 | |
| HSA-dF (Hz) | | | | | | | | | | |
| 12.3 nM Anti-Has- | | | 77.7 | 12.3 | nM | | | 74.5 | 2.50E−03 | |
| HSA-dF (Hz) | | | | | | | | | | |
| 4.1 nM Anti-HAS- | | | 57.8 | 4.1 | nM | | | 51.1 | 9.04E−04 | |
| HSA-dF (Hz) | | | | | | | | | | |
| 1.4 nM Anti-HSA- | | | 31.8 | 1.4 | nM | | | 22.9 | 3.78E−04 | |
| HSA-dF (Hz) | | | | | | | | | | |
| | 1.95E+05 | 1.06E−04 | | | | 1.84E+09 | 5.44E−10 | | | 3.42 |

### Example 6

Quartz Crystal Resonance Sensing Apparatus

Quartz crystal experiments were carried out on an instrument as follows. AT-cut Quartz crystals having a mesa structure with two resonators etched into the substrate were coated with a titanium adhesion layer (5 nm) and gold (200 nm) using conventional evaporation techniques. The apparatus is illustrated schematically in FIG. 6.

The sensor substrate is docked into a temperature controlled (25° C.) flow cell which has a microfluidics polydimethylsiloxane (PDMS) insert that enables the delivery and removal of liquids using computer controlled syringe pumps (2, 4), one (2) for buffer, and one (4) for sample. The insert is replaceable and has two versions which can be used to address the two sensors separately (6) or in common (8) with reagent in solution. A multiway Rheodyne valve (10) is used to provide either buffer or reagent to the sensors. The buffer and sample macrofluidic system comprises two Hamilton syringe pump units, a pair of Y-connectors (12, 14), an isolated air compression (or ‘thumper’) valve (16), a sample load/inject multiway valve (10) and a flow cell sensor selector valve (18). The two syringes (typically 500 μl glass syringes) on the buffer pump unit dispense alternately to maintain a constant buffer flow. The sample pump (4) comprises a single 5 ml syringe to draw the sample to be injected into the sample loop and is then injected by diversion of the running buffer through the sample loop.

The multiway valve (10) is used either to direct buffer flow immediately to the flow cell selector valve, or to divert it through the sample loop to push a preloaded sample onwards to the flow cell sensor selector valve (18).

A network analyser is used to drive the dual quartz crystal sensors sequentially over a frequency range which includes the resonant frequencies of approximately 13.9 MHz. The impedance of the sensors is measured as a function of frequency and the resonant frequency determined by fitting of the impedance vs. frequency data to an equivalent circuit model for the sensor. The frequency shift is monitored in real time at a sampling rate of 10 Hz.

Deposition of Polymers

In the examples described hereinafter, the two sensors were first coated together with polymer by single cell addressing. Following completion of the coating, the sensors were then washed thoroughly in buffer to remove all trace of liberated PDMA, and then replaced in the cell.

Deposition of Protein

The cells were then addressed individually with HSA to form the surface bearing the first member of a specific binding pair (the second member being an antibody to HSA) and BSA to form the reference surface respectively. To measure the immobilisation of anti-HSA on HSA the two sensors were then used in single addressing mode again and anti-HSA solution was flowed over both sensors. The change in frequency in real time for the sample and reference channels were recorded. The examples were repeated using different concentrations of anti-HSA analyte. The frequency shift of the sample sensor was corrected for the background shift of the reference sensor, and this corrected value was taken as a measure of attached anti-HSA. Analysis was performed as described below.

The resonator was primed with a constant flow of Milli Q ultrapure water, at a flow rate of 120 μl/min, then cleaned with 2×5 min injections of a solution of 100 mN NaOH/1% Triton X-100. Immediately following this injection, the resonator was exposed to 2×7 min injections of a 1% w/v solution of Dextran T70-PDEA (15% —AKU34-178) in water, then 2×30 min injections of a solution of bromoacetic acid (1.75 g) in 2M NaOH (15 ml) to convert hydroxyl groups on the polymer to carboxylmethyl groups, according to the procedure of Reference 40.

The resultant modified polymer was then activated by a 5 min injection of a solution of EDC (200 mM) mixed with NHS (50 mM), then exposed to a solution of human serum albumin (HSA, 1 mg/ml in 10 mM NaOAc buffer, pH 4.5). Residual activated NHS esters were then capped by a 4 min injection of IM ethanolamine, pH 8.5.

TABLE 3
Frequency and resistance changes for deposition,
activation and coupling of protein using Dextran
T70-PDEA (15% - AKU34-178) as measured by a QCM sensor.

| | Polymer | Bromoacetic | HSA | Ethanolamine |
|---|---|---|---|---|
| | deposition | acid activation | coupling | capping |
| Frequency shift | −472 | −196 | −1115 | −19.6 |
| (Hz) | | | | |
| Resistance | 11.5 | 29.7 | 15.7 | 1.5 |
| change (Ohm) | | | | |

The amount of polymer to become bound to the gold surface when applied by the method of Comparative Examples 1 and 2 and Example 1 varied according to the nature of the leaving group. Using T70 dextran with 6% of hydroxy group derivatised by —CONHCH₂CH₂SSR2 where R2 was 2-pyridyl, CH₂CH₂CO₂H or CH₂CH₂NH₂, it was found that use of the 2-py leaving group produced deposition as measured by SPR of about 2700 pg/mm² whereas the other two leaving groups resulted in deposition of about 1200 pg/mm². (Deposition on commercial BIACORE®™ 2000 SPR biosensor). Increasing the derivatisation of the dextran T70 to 10% and 15% with CONHCH₂CH₂SS-2-Py side chains further increased deposition to about 3500 pg/mm² and 5000 pg/mm² respectively. When dextran T500 derivatised to 5% with side chains —CONHCH₂CH₂SSR2, the deposition when R2 was 2-py was approximately 2500 pg/mm² and when R2 was CH₂CH₂NH₂ was approximately 250 pg/mm².

Dextran T70 PDEA (6% substitution, 0.1-2% w/v in water) was injected from t=10 minutes to t=17 minutes on a gold quartz resonator oscillating at 14.3 MHz in a flow cell formed by clamping the crystal between two O-rings. The signal was determined using a network analyser. A frequency change of about 800 Hz was observed due to adsorption of the polymer on to the gold surface.

In an SPR experiment a similar layer of PDEA on T70 was found to produce a response of about 3900 RU. This signal was reduced by about 800 RU on washing with 100 mM NaOH but was thereafter stable to washing with 100 mM NaOH, 1% Triton X-100, 1 mM cysteine and 1 nM dithiothreitol (DTT). This demonstrates that the polymer was effectively bound to the metal.

The loading of T70 PDEA and T500 PDEA onto gold on a SPR sensor was measured in real time and compared to T10 propionic acid and T70 propionic acid analogues. The polymers produced signals indicating about 2.5 to 3 times as much binding occurred with the PDEA derivatised polymers as with the acid derivatised polymers. The T500 PDEA polymer was particularly stable to washing with 100 mM NaOH and Triton X-100.

The SPR response of T10-propionic acid, T70-propionic acid, T500-PDEA and T70-PDEA to treatment with undiluted human serum over 5 minutes at a flow rate of 10 μl/ml were determined. The non-specific deposition was about 1900, 1500, 600 and 150 RU respectively.

The SPR responses to binding of an anti-HSA mouse monoclonal antibody at 111 nM to either BSA or HSA immobilised on two different polymers (6% and 15% dextran T70 PDEA) were measured. At 5 minutes to 10 minutes a stable value of about 600 RU and 800 RU was obtained for the 6% and 15% polymers which had immobilised HSA. No change from base line was observed for polymers which had immobilised BSA. After 10.2 minutes the surface was regenerated by treating with 10 mM glycine pH 2.0 returning the positive signal to zero. Repetition of this cycle ten times showed little or no variation, demonstrating the excellent stability of the system. HSA immobilised on dextran T70 PDEA on gold on a SPR biosensor was exposed to anti-HSA monoclonal antibody at serial three fold dilutions from 333 to 4.1 nM. The results at 5 to 10 minutes was about 410, 320, 210 and 75 RU allowing the preparation of a calibration curve to allow assay of unknown concentrations of the antibody.

### Example 7

### Detection of Small Molecules by SPR

15% PDEA substituted dextran surfaces (AKU34-178) were prepared on Biacore chips as follows.

Au SPR chips were cleaned by treatment with plasma ashing: (Argon plasma, 5 Pa, 100 W, 15 sec). Immediately after plasma ashing, the chips were incubated in a humid environment at room temperature in 1% w/v AKU34-178 (fresh aliquot from —80° C. freezer) in water placed carefully over SPR chip (held by surface tension). After 18 h the chips were rinsed exhaustively in water then immersed for 20 h in a solution of 1.75 g bromoacetic acid (12.5 mmol) in 13.5 ml of 2M NaOH. The treatment with bromoacetic acid converts the hydroxyl groups of the dextran polymer to reactive COOH groups which can subsequently be conjugated to members of specific binding pairs (e.g. antibodies). After this treatment the chips were again washed exhaustively with ultrapure water, then with spectroscopic grade ethanol, then with water again. After washing the chips were blown dry under a flow of nitrogen, mounted using ‘double-sided sticky tape’ on Biacore plastic cassettes. The cassettes were again blown dry under nitrogen and then stored in 50 ml Falcon tubes at room temperature under argon until required.

4 flow cells having polymer treated surfaces were prepared. Flow cells (FC) 1, 2 & 3—were all activated by exposure for 7 min to EDC/NAS (200 mM/50 mM). FC 3 only was then treated for 7 min with 50 μg/ml mouse anti-biotin IgG in 10 mM NaOAc buffer pH 4.5. FC 2 only was coupled to 50 μg/ml an irrelevant mouse monoclonal IgG in 10 mM NaOAc buffer pH 4.5 (7 minute exposure). FC 1, 2, 3—were capped by treatment for 5 min with ethanolamine (1 M, pH 8.5). FC 4 comprised untreated polymer.

| Surface | Treatment |
|---|---|
| FC 1 | Activated polymer, capped (blank) |
| FC 2 | Activated polymer, coupled to irrelevant (non biotin-specific) |
| | mouse IgG, capped (non-specific control) |
| FC 3 | Activated polymer, coupled to specific mouse anti-biotin IgG, |
| | capped (experimental) |
| FC 4 | Unactivated polymer (blank) |

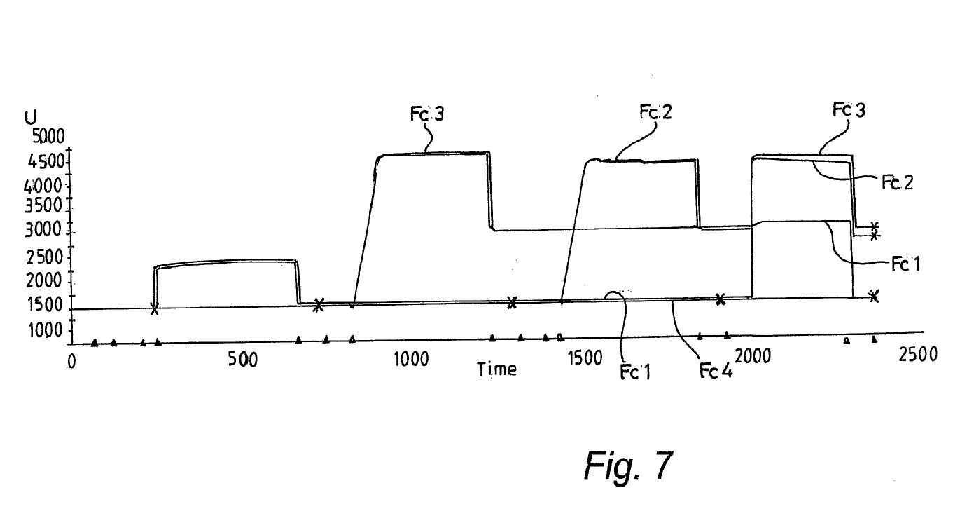

The plots in FIG. 7 show graphs of response (in arbitrary response units), against time (in seconds) for these immobilisation stages in the treatment of each surface.

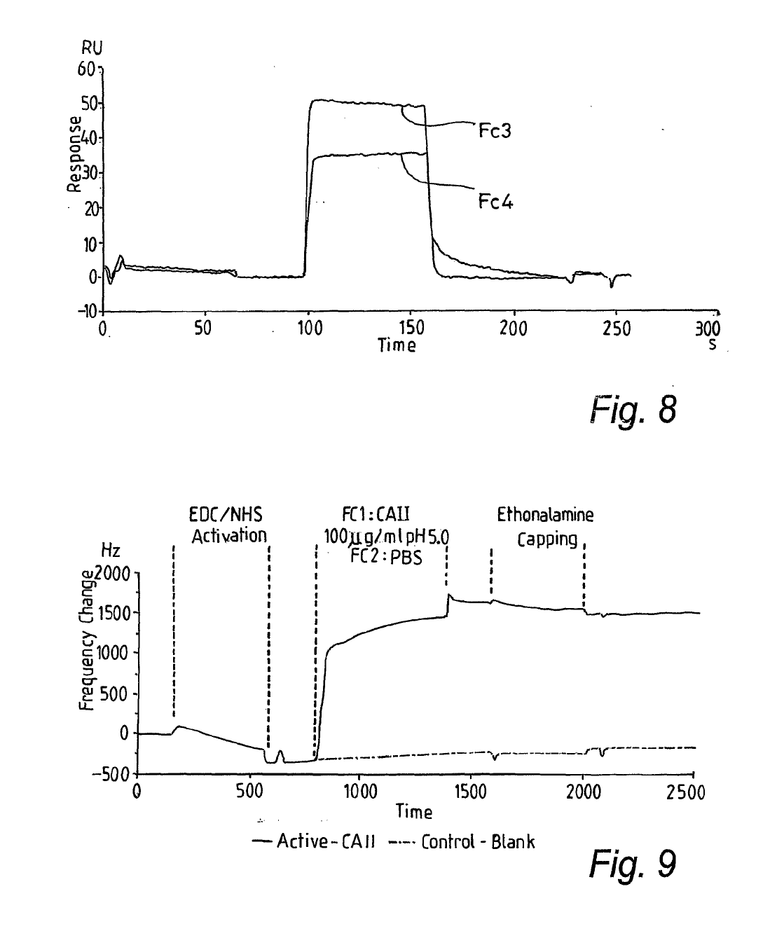

Biotin at concentration of 10 μM was then passed over all four surfaces. The response to binding of biotin of the anti-biotin surface in FC3 and the underivatised control surface of FC 4 is shown in FIG. 8.

### Example 8

### Detection of Small Molecules by QCRS

15% PDEA substituted dextran surfaces (AKU 34-178) were prepared as previously, residual PDEA groups capped, and the OH groups on dextran converted to COOH reactive groups as in Example 7. The surface was activated by EDC/NHS and exposed to carbonic anhydrase II (CAII). Reaction took place between the COOH and amine groups of the CA II and produced a frequency shift of 1800 Hz due to attached protein. The control surface was activated and capped with ethanolamine. Typical results for the preparation of the surfaces are shown in FIG. 9, which is a graph of frequency change (Hz) against time (seconds).

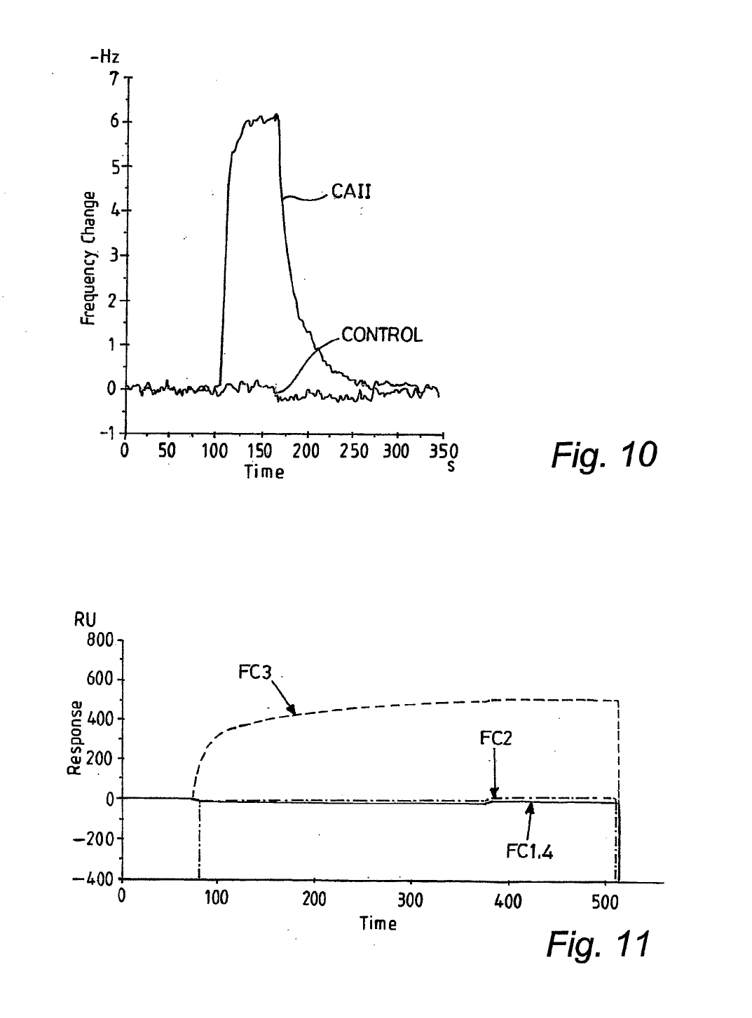

The CAII surface was then used to capture 4-carboxybenzenesulfonamide (CBS 25 μm in running PBSS buffer+0.25% MeOH). Attachment of mass to the surface, sufficient to cause a frequency shift of 6 Hz, was observed. Typical results are shown in FIG. 10 (graph of frequency change in Hz against time in seconds).

The same CAII surface was used to detect 12.5 μM 4-carboxybenzenesulfonamide, and 12.5 μM 5-(dimethylamino)-1-naphthalenesulfonamide. A shift of 2.5 Hz and 1.2 Hz respectively was observed, with a signal to noise ratio of ca 10.

### Example 9

### Detection of Proteins by SPR

The surfaces described in Example 7 were exposed to 10 μg/ml biotinylated BSA and the binding is shown in FIG. 11, which is a graph of response (in arbitary Response Units) against time (in seconds). Here, flow cell (FC)3 shows binding of the biotinylated BSA to mouse anti-biotin IgG, FC2 shows non-specific binding to the mouse IgG control surface, FC1 shows binding to the the capped polymer and FC4 shows binding to the free polymer.

### Example 10

### Detection of Proteins by SPR

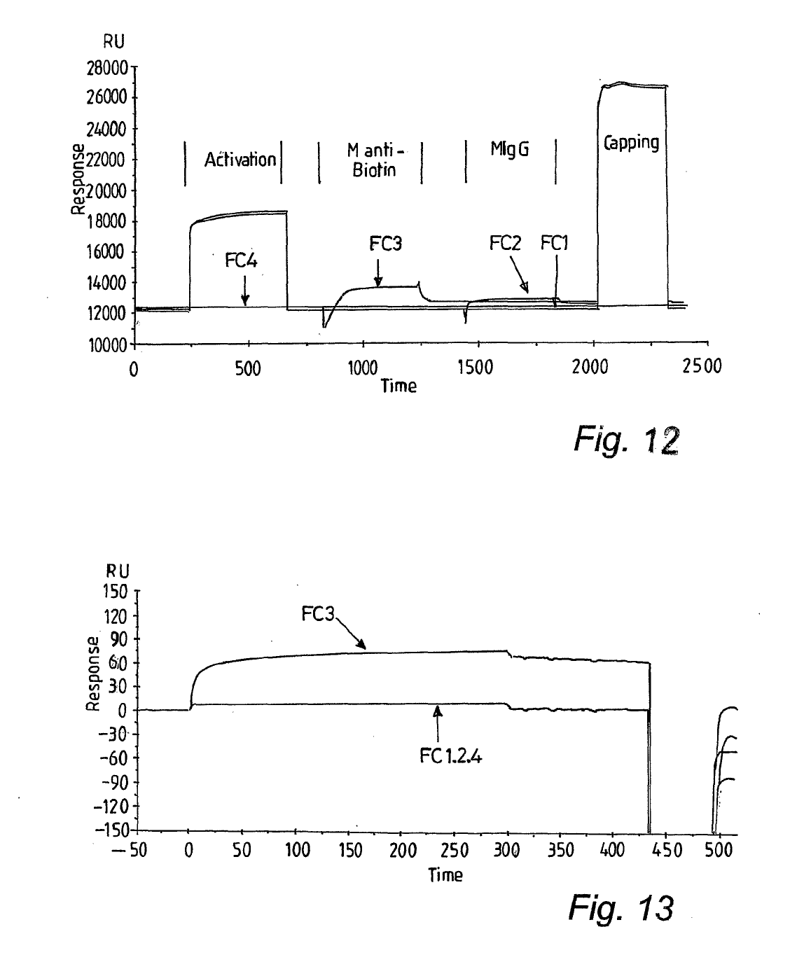

Dextran T70-COOH-PDEA (428018) was deposited and mouse anti-biotin IgG was immobilised using the same procedure as in Example 7. The measurement of the immobilisation is shown in FIG. 12, which again is a graph of response (RU) against time (seconds).

The surfaces were then exposed to 10 μg/ml biotinylated BSA and the binding is shown in FIG. 13. Specific binding occurs in FC3, and minimal non-specific binding is shown in FC1, 2 and4.

### Example 11

### Deposition of Thiosulphone Polymers—SPR

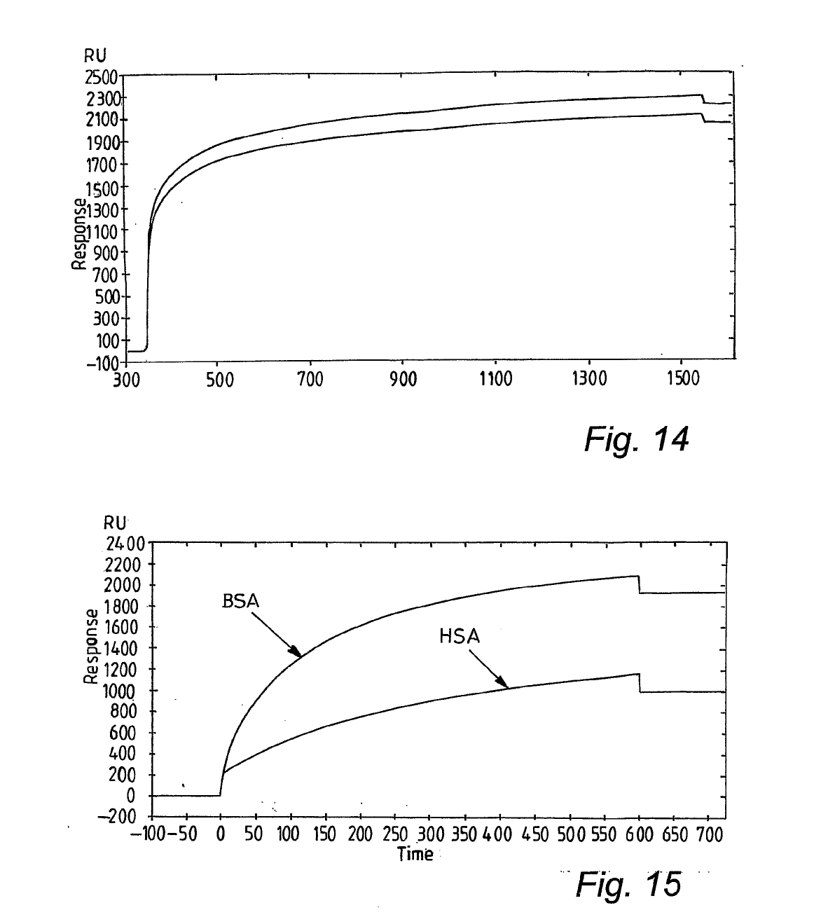

Proteins, reagents, gold surfaces suitable for SPR analysis, and antibodies where prepared as for Example 2. An alternate leaving group polymer, Dextran-T70 thiosulfone (428034 from Example 1) was prepared as a 1 mg/ml solution and deposited on the gold surface of an SPR sensor chip as described in Example 2. This resulted in deposition in the order of 2200 RU (corresponding to ca. 2.2 ng/mm² polymer bound to the surface). Duplicate injections of this polymer yielded the results shown in FIG. 14 (RU against time, seconds).

### Example 12

### Coupling of Proteins to Thiosulphone Polymers—SPR

To the thiosulfone-polymer-coated (428034) gold surfaces of Example 11 was coupled human serum albumin (HSA) or bovine serum albumin (BSA) as described in Example 3. The proteins couple directly to the thiosulphone via their sulfhydryl groups, and the result is shown in FIG. 15 (graph of RU against time).

### Example 13

### Binding of Antibodies to Protein-captured Protein Receptors on Thiosulphone Polymers—SPR

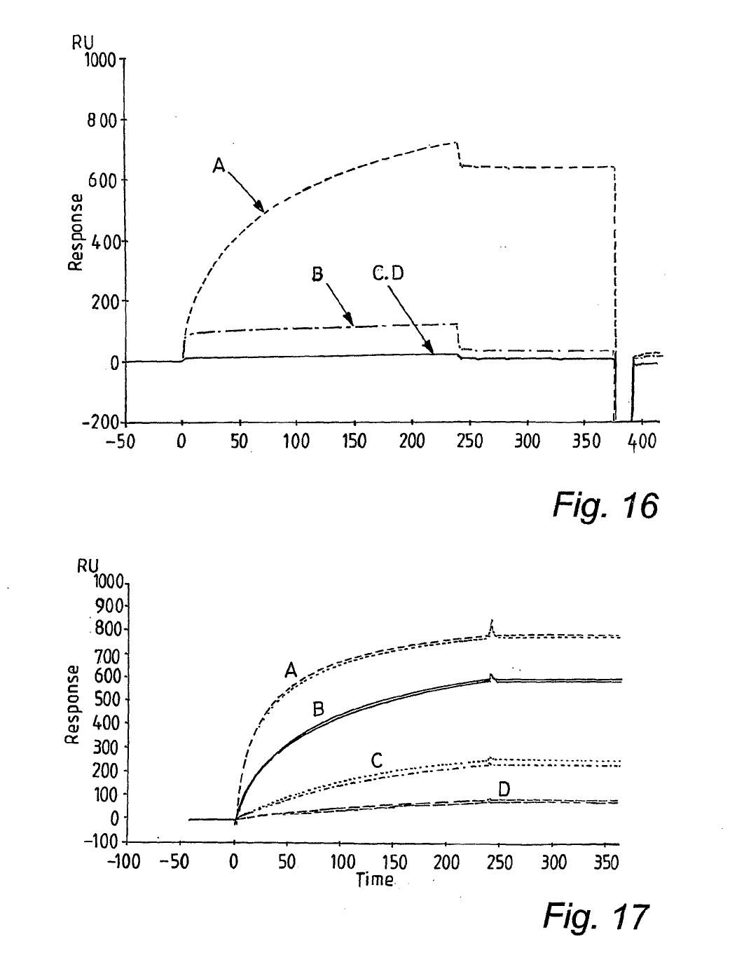

Anti-HSA IgG was prepared and diluted and then passed over both protein-coupled polymer surfaces of Example 12 using the method described in Example 5, except that regeneration of the free protein receptor was effected by injection of a solution of 10 mm HCl (5 ml, 40 ml/min). In addition a control antibody, normal mouse IgG, was injected at a concentration of 11 nM over both surfaces. FIG. 16 (graph of RU against time) shows the result of binding of anti-HSA to the HSA surface in curve A; of antiHSA to the BSA surface in curve B; of antimouse IgG binding to the HSA surface in curve C, and of mouse IgG binding to the BSA surface in curve D.

Duplicate threefold serial dilutions of anti-HSA were injected over the HSA and BSA surfaces. The results corrected by subtraction of the control BSA binding curve from the HSA binding curve are shown in FIG. 17 (graph of RU against time; A: 333 nM HSA; B: 111 nM; C: 37 nM; D: 12.3 nM.

References (the content of all the following citations is incorporated herein by reference):
Ref. 1 Beyer et al., Thin Solid Films (1996), 285, 825-828
Ref. 2 Shena et al., Science, 270 (1995), 467-470
Ref. 3 Huang et al., Langmuir (2001), 17, 489-498
Ref. 4 Sackmann, Science 271 (1996), 43-47
Ref. 5 EP 254575
Ref. 6 Ruiz-Taylor et al., Proc. Natl. Acad. Sci. USA (2001) 98, 852-7
Ref. 7 Huang et al., Langmuir (2001), 18, 220-230
Ref. 8 Kenausis et al., J. Phys. Chem. (2000), B104, 3298-3309.
Ref. 9 Prime et al., J. Am. Chem. Soc. (1993), 115, 10714-10721.
Ref. 10 Ostuni et al., Langmuir (2001), 17, 5605-5620
Ref. 11 US 2002/0127577A1
Ref. 12 U.S. Pat. No. 5,242,828
Ref. 13 Storri et al., Biosensors of Bioelectronics (1998), 13, 347-357
Ref. 14 Tombelli et al., Biosensors of Bioelectronics (2002), 17, 929-936
Ref. 15 Frazier et al., Biomaterials (2000), 21, 957-966
Ref. 16 Xia et al., Langmuir, (2002), 18, 3255-3262
Ref. 17 Lenk et al., Macromolecules (1993), 26, 1230-1237
Ref. 18 Nakayama, Langmuir (1998) 14, 3909-3915
Ref. 19 Schlenhoff et al., Macromolecules (1995), 28, 4290-4295
Ref. 20 Sun et al., Langmuir (1993) 9, 3200-3207
Ref. 21 Sun et al., J. Vacuum Sci. & Tech. (1994), 12, 2499-2506
Ref. 22 U.S. Pat. No. 6,413,587
Ref. 23 EP 1035218A1
Ref. 24 US 2002 00126
Ref. 25 Burgener et al., Bioconjugate Chem. (2000), 11, 749-754
Ref. 26 Trost et al., Tetrahedron Lett. (1981), 22, 1287-1290
Ref. 27 Yagupolskii et al., ZOK (1968), 38, 2426
Ref. 28 Yagupolskii et al., ZOK (1968), 35, 2509-2513
Ref. 29 Uemura et al., Perkin Trans. (1990), 1, 1697-1703
Ref. 30 Uemura et al., Perkin Trans. (1986), 1, 1983-1987
Ref. 31 Krief et al., J. Chem. Soc., Chem. Commun. (1985), 571-572
Ref. 32 Weidner et al., J. Med. Chem. (1972), 5, 564-567
Ref. 33 Thomas et al., J. Org. Chem. (1961), 26, 3780-3783
Ref. 34 Hart et al., Tetrahedron Lett. ((1985), 32, 3879-3882
Ref. 35 Reich, Oxidation in Organic Chemistry, Trahanovsky, Ed., Academic Press Inc., NY (1978), Vol. 5, Part C, pp 1-35
Ref. 36 Appel, Angew, Chem. Int. Ed. Engl. (1975), 14, 801-811
Ref. 37 Lindgren, J. Chem. Soc. (1980), 16, 24
Ref. 38 Uemura et al., J. Chem. Soc., Perkin Trans. (1985), 1, 471-480
Ref. 39 Mukhopadhyay et al., Chem. Commun. (2004), 4, 472-473

Ref. 40 Lofas et al., J. Chem. Soc., Chem. Commun. (1990), 526-1528

APPENDIX 1
Summary of Neutral Reactive Groups

| Reagent | | Functional | Activation | | | Coupling | Appendix |
|---|---|---|---|---|---|---|---|
| for activation | Matrix | group | conditions | Activated structure | Ligand | pH range | Ref. |
| CNBr (Cyanogen bromide) | Polyol (esp. polysacch | —OH | aq./buffer | 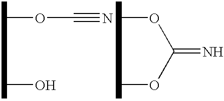 | —NH₂ | 7-8.5 | [1-3] |
| CDAP 1-Cyano-(4- dimethylamino) pyridinium tetrafluoro borate) | Polyol (esp. polysacch | —OH | aq./organic | 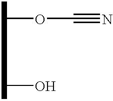 | —NH₂ | 7-8.5 | [2, 3] |
| DSC (Disuccinimidyl carbonate) | Polyol (esp. polysacch | —OH | Organic | 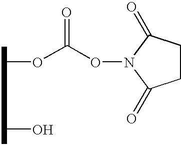 | —NH₂ | 6-8 | [4] |
| CDI | Polyol | —OH | Organic | Various | —NH₂ | 8-10 | [5] |
| (Carbonyl- | (esp. | | | | | | |
| diimidazole) | agarose.) | | | | | | |
| Tosyl chloride | Polyol (esp. agarose.) | —OH | Organic | 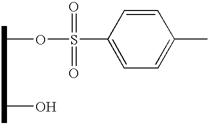 | —NH₂—SH | 9-10 | [6] |
| Tresyl chloride | Polyol (esp. agarose.) | —OH | Organic | 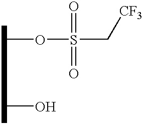 | —NH₂—SH | 8-9 | [6] |
| Bisoxiranes | Polyol | —OH | aq. pH 13-14 aq. pH 8-10 | 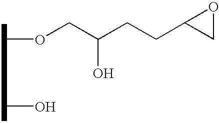 | —OH —NH₂, SH | 11.5-13 8-11 | [7] |
| Epichlorohydrin | Polyol | —OH | aq. pH 13-14 | 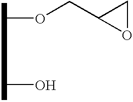 | —OH —NH₂, SH | 11.5-13 8-11 | [8, 9] |
| Divinylsulfone | Polyol | —OH | aq. pH 13-14 | 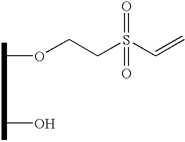 | —OH —NH₂, SH | 10.5-12 8-11 | [10] |
| Carbodiimides | Polyol | —COOH | aq. | 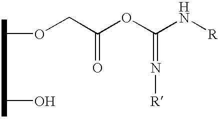 | —NH₂ | 5 | [11-13] |
| NHS/EDC | Polyol | —COOH | aq. | 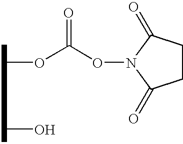 | —NH₂ | 5-9 | [14] |
| Matrix thiol | Polyol | —SH | aq. | | —CH═CH—, | 8-10 | [11] |
| | | | | | ═CO, —CNH | | |
| Thiol-disulfide exhange | Polyol | —SH | aq. | 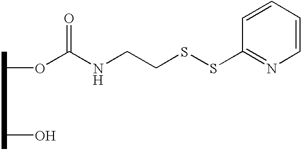 | —SH | 2-9 | [15] |
| Glutaraldehyde | Polyamide | —CONH₂ | aq. | 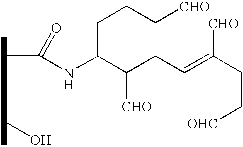 | —NH₂ | 7 | [16] |
| Hydrazine | Polyamide | —CONH₂ | NaNO₂/HCl | | —CHO, —CO | 7-9 | [17] |
| Silyl oxirane | Silica | —SiOH | | 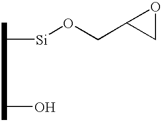 | —NH₂, —SH | 8 | [18] |
| Isocyanide | Various | —COOH —NH₂═CO, —NC | aq. pH 6.5 | 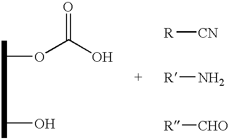 | —NH₂, —COOH, —COH, ═CO | 6.5 | [19] |

Appendix Refs
1 Prorate, J., Asperg, K., Drevin, H. and Axen, R. (1973) J. Chromatogr. 86, 53
2 Kohn, J. and Wilchek, M. (1984) Applied Biochemistry and Biotechnology 9, 285-305
3 Kohn, J. and Wilchek, M. (1983) Febs Letters 154, 209-210
4 Wilchek, M. and Miron, T. (1985) Applied Biochemistry and Biotechnology 11, 191-193
5 Hearn, M. T. W. (1987) in Methods Enzymol., vol. 135 (Mosbach, K., ed.), pp. 102-117, Academic Press, New York
6 Nilsson, K. and Mosbach, K. (1987) in Methods Enzymol., vol. 135 (Mosbach, K., ed.), pp. 65-78, Academic Press, New York
7 Sunberg, L. and Porath, J. (1974) J. Chromatogr. 90, 87
8 Porath, J. and Fornstedt, N. (1970) J. Chromatogr. 51, 479
9 Axen, R., Carlsson, J., Janson, J. C. and Porath, J. (1971) Enzymologia 41, 359-64
10 Porath, J. (1974) in Methods Enzymol., vol. 34 (Jakoby, B. and Wilchek, M., eds.), pp. 13-30, Academic Press, New York
11 Robinson, D., Phillips, N. C. and Winchester, B. (1975) FEBS Lett 53, 110-2
12 Anttinen, H. and Kivirikko, K. I. (1976) Biochim Biophys Acta 429, 750-8
13 Marcus, S. L. and Balbinder, E. (1972) Anal Biochem 48, 448-59
14 Cuatrecasas, P. and Parikh, I. (1972) Biochemistry 11, 2291-9
15 Brocklehurst, K., Carlsson, J., Kierstan, M. P. and Crook, E. M. (1973) Biochem J 133, 573-84
16 Guesdon, J. L. and Avrameas, S. (1976) J Immunol Methods 11, 129-33
17 Inman, J. K. (1974) in Methods Enzymol., vol. 34 (Jakoby, B. and Wilchek, M., eds.), pp. 30, Academic Press, New York
18 Ohlson, S., Hansson, L., Larsson, P. O. and Mosbach, K. (1978) FEBS Lett 93, 5-9
19 Goldstein, L. (1987) in Methods Enzymol., vol. 135 (Mosbach, K., ed.), pp. 90-102, Academic Press, New York

1. A polymer comprising covalently bound side chains of the formula —X—Y—Z—R wherein X is a spacer group; Y is a sulphur, selenium or tellurium atom; Z is a sulphur, selenium or tellurium atom any of which may be bonded to one or two oxygen atoms; and wherein R is any suitable moiety such that —Z—R constitutes a leaving group.

2. A polymer according to claim 1, wherein R is a moiety such that the conjugate acid HZR has a pKa of less than 8.

3. A polymer according to claim 1, wherein R is a moiety such that the conjugate acid HZR has a pKa of less than 6.

4. A polymer according to claim 1, wherein R is a moiety such that the conjugate acid HZR has a pKa of less than 4.

5. A polymer according to claim 1, wherein Y is S or Se, preferably S.

6. A polymer according to claim 1, wherein Z is S, SO, or SO₂.

7. A polymer according to claim 6, wherein Z is S or SO₂.

8. A polymer according to claim 1, wherein R comprises one of the following: an unsaturated group conjugated to an electron withdrawing group; an aromatic group; a heteroaromatic group; and an electrophilic group.

9. A polymer according to claim 8, wherein the electron withdrawing group comprises one or more of the following: lower alkyloxycarbonyl; nitrile; nitro; lower alkylsulphonyl; and trifluoromethyl.

10. A polymer according to claim 8, wherein the aromatic group comprises: optionally substituted phenyl, wherein there may be up to three substituents selected from nitro, trifluoromethyl, nitrile, lower alkyloxylcarbonyl or othyer electron withdrawing groups.

11. A polymer according to claim 8, wherein the heteroaromatic group comprises a 5- or 6-membered ring, optionally fused to the residue of a phenyl ring or a further 5- or 6-membered heteroaromatic ring, and wherein the said heteroaromatic ring or further heteroaromatic ring may optionally be substituted by one or two lower alkyl, phenyl, ═O, ═S, trifluoromethyl, nitro or nitrile groups.

12. A polymer according to claim 1, wherein the moiety —Z— R is derived from an aromatic thiol, a heteroaromatic thiol or their thione tautomers.

13. A polymer according to claim 12, wherein the moiety —Z—R is derived from the group consisting of:
imidazole; pyrrolidine-2-thione; 1,3-imidasolidine-2-thione; 1,2,4-triazoline-3(5)-thione; 1,2,3,4-tetrazoline-5-thione; 2,3-diphenyl-2,3-dehydrotetrazolium-5-thione; N(1)-methyl-4-mercaptopiperidine; thiomorphyline-2-thione; thiocaprolactam; pyridine-2-thione; pyrimidine-2-thione; 2-thiouracil; 2,4-dithiouracil; 2-thiocytosine; quinoxazoline-2,3-dithione; 1,3-thiazoline-2-thione; 1,3-thiazolidine-2-thione; 1,3-thiazolidine-2-thione-5-one; 1,3,4-thiadiazoline-2,5-dithione; 1,2-oxazolidine-2-thione; benz-1,3-oxazoline-2-thione; 1,3,4-oxadiazoline-2-thione and analogues in which the sulphur is replaced by selenium or tellurium.

14. A polymer according to claim 1, wherein R is the 2-pyridyl group.

15. A polymer according to claim 1, wherein —Z—R is the —S-2-pyridyl group.

16. A polymer according to claim 1, wherein —Y—Z—R is the —S-2-Z-pyridyl group.

17. A polymer according to claim 1, wherein the spacer X comprises an alkylene or phenyl group which may be unsubstituted or substituted by one or more lower alkyloxy, halo, oxo, trifluoromethyl, nitrile or other groups which do not interfere with the formation and use of the —Y—Z—R moiety.

18. A polymer according to claim 1, wherein the spacer X comprises a linking moiety through which the side chain is attached to the rest of the polymer, the linking moiety being selected from the group consisting of: —O—, —O—CO—, —O—CO—O—, —NH—, lower alkyl substituted —NH—, —O—CO—NH—, and lower alkyl N-substituted —O—CO—NH—.

19. A polymer according to claim 1, wherein the spacer group is of the formula —A—B, wherein B is an unsubstituted or substituted alkylene or phenyl group which may be unsubstituted or substituted by one or more lower alkyloxy, halo, oxo, trifluoromethyl, nitrile or other groups which do not interfere with the formation and use of the —Y—Z—R moiety and A is a linking moiety being selected from the group consisting of: —O—, —O—CO—, —O—CO—O—, —NH—, lower alkyl substituted —NH—, —O—CO—NH—, and lower alkyl N-substituted —O—CO—NH—.

20. A polymer according to claim 17, wherein the spacer X comprises a moiety B which is a lower alkylene group, optionally interrupted by an oxygen atom, carboxyl group or carboxyloxy group.

21. A polymer according to claim 20, wherein the spacer X comprises a moiety B which is a straight chain alkylenyl group —(CH₂)ₙ— wherein n is 1-4, preferably 2.

22. A polymer according to claim 1, wherein X is —CO—NH—CH₂—CH₂, Y is S, Z is S and R is 2-pyridyl.

23. A polymer according to claim 1 which is hydrophilic.

24. A polymer according to claim 1 which is neutral.

25. A polymer according to claim 1 wherein the —X—Y—Z—R side chains are attached to a molecule selected from the group consisting of: dextran; hyaluronic acid; sepharose; agarose; nitrocellulose; polyvinyl alcohol; partially hydrolysed polyvinylacetate or polymethylmethacrylate; carboxymethyl cellulose; and carboxymethyl dextran.

26. A polymer according to claim 1, wherein the —X—Y—Z—R side chains are attached to a molecule derived from sugar monomeric units.

27. A polymer according to claim 1, wherein in addition to —X—Y—Z—R side chains, the polymer also comprises side chains according to the formula —X—Y—Z—R1, wherein X, Y and Z are as defined in claim 1 and R1 is a member of a specific binding pair.

28. A substrate which has reacted with a polymer in accordance with claim 1, such that at least some of the —Z—R groups of the side chains are displaced and the polymer becomes covalently attached to the substrate via —X—Y— side chains.

29. A substrate according to claim 28, wherein at least part of the substrate is coated with a polymer according to claim 1, the polymer being covalently attached to the substrate via —X—Y— side chains, and wherein at least some of —Z—R groups of the side chains are not displaced.

30. A substrate according to claim 28, comprising a metal surface.

31. A substrate according to claim 30, wherein the metal surface comprises gold, silver, platinum, palladium, nickel, chromium, titanium, copper or an alloy of any thereof.

32. A substrate according to claim 28, wherein the substrate forms part of a biosensor.

33. A substrate according to claim 28, wherein the substrate comprises a quartz crystal or other piezoelectric material.

34. A substrate according to claim 28, comprising a polymer in accordance with claim 1 covalently attached to a metal surface, the metal surface being present on a solid support.

35. A substrate according to claim 34, comprising an adhesion layer disposed between the metal surface and the solid support.

36. A biosensor comprising a substrate in accordance with claim 28.

37. A biosensor according to claim 36, wherein the biosensor is selected from the group consisting of SPR biosensors and acoustic biosensors.

38. A method of indirectly attaching a moiety to a substrate, the method comprising the step of reacting the substrate with a polymer in accordance with claim 1, wherein the polymer includes side chains which comprise the moiety to be indirectly attached to the substrate.

39. A method of indirectly attaching a moiety to a substrate, the method comprising the steps of: reacting the substrate with a polymer in accordance with claim 1, said reaction displacing some, but not all, of the —Z—R groups from the side chains of the polymer, such that the polymer becomes attached to the substrate by —X—Y groups; and contacting the attached polymer with a reagent comprising the moiety to be indirectly attached to the substrate, so as to cause the moiety to become attached to the polymer.

40. A method according to claim 39, wherein the reagent reacts with the undisplaced —Z—R groups present on the attached polymer.

41. A method according to claim 39, wherein the attached polymer is further modified by a chemical before contacting with the reagent which adds the moiety to be indirectly attached to the substrate.

42. A method according to claim 41, wherein a hydroxy, carboxy, epoxy, or amino group present on the polymer is used to attach the moiety.

43. A method according to claim 38, wherein the moiety is a member of a specific binding pair.

44. A polymer according to claim 1, wherein in addition to —X—Y—Z—R side chains, the polymer also comprises side chains according to the formula —X—Y—Z—R1, where X, Y and Z are as defined in claim 1 and R₁ is a reactive moiety to which a member of a specific binding pair can become attached.

45. A polymer according to claim 28, wherein the reactive moiety comprises an amino, hydroxy, carboxy or epoxy group.

46. A method according to claim 41, where the reactive moiety is a member of a specific binding pair.
